(19) United States
(12) Patent Application Publication
(10) Pub. No.: US 2018/0078129 A1
(43) Pub. Date: Mar. 22, 2018

(54) IMAGING DEVICE AND METHOD FOR IMAGING SPECIMENS

(71) Applicants: NANYANG TECHNOLOGICAL UNIVERSITY, Singapore (SG); SINGAPORE HEALTH SERVICES PTE LTD, Singapore (SG)

(72) Inventors: Murukeshan Vadakke Matham, Singapore (SG); Shinoj Vengalathunadakal Kuttinarayanan, Singapore (SG); Xun Jie Jesmond Hong, Singapore (SG); Baskaran Mani, Singapore (SG); Tin Aung, Singapore (SG)

(73) Assignees: Nanyang Technological University, Singapore (SG); Singapore Health Services PTE Ltd., Singapore (SG)

(21) Appl. No.: 15/553,181

(22) PCT Filed: Feb. 24, 2016
(86) PCT No.: PCT/SG2016/050089

§ 371 Date: Aug. 24, 2017

(30) Foreign Application Priority Data
Feb. 25, 2015 (SG) 10201501380W

(51) Int. Cl. A61B 3/00; A61B 3/14; A61B 3/13; G02B 21/00; G02B 27/09

(52) U.S. Cl. CPC A61B 3/0008; A61B 3/14; A61B 3/13; G02B 21/0028; G02B 5/001; G02B 21/0048; G02B 27/0927; G02B 27/0955; G02B 21/0076; G02B 21/0032

(57) ABSTRACT

According to various embodiments, there is provided an imaging device including a Bessel beam generator configured to provide a Bessel beam; a scanning mirror configured to scan the Bessel beam across a two-dimensional plane; a scan lens configured to receive the Bessel beam from the scanning mirror, a centre of the scan lens being at least substantially a focal length of the scan lens away from the scanning mirror; an illumination tube lens configured to receive the Bessel beam from the scan lens, a centre of the illumination tube lens being at least substantially a sum of the focal length of the scan lens and a focal length of the illumination tube lens away from the centre of the scan lens; an illumination objective lens positioned in direct line-of-sight to a specimen, the illumination objective lens configured to receive the Bessel beam from the illumination tube lens and further configured to illuminate the specimen with the Bessel beam, wherein a centre of the illumination objective lens is at least substantially the focal length of the illumination tube lens away from the centre of the illumination tube lens; and a detection optics arrangement configured to receive a reflected beam (emitted fluorescence beam) from the specimen.

### CROSS-REFERENCE TO RELATED APPLICATIONS

This application claims the benefit of Singapore Patent Application number 10201501380W filed 25 Feb. 2015, the entire contents of which are incorporated herein by reference for all purposes.

### TECHNICAL FIELD

The present invention relates to imaging devices and methods for imaging specimens.

### BACKGROUND

Glaucoma is an eye disease that may result in the loss of sight by damaging the optic nerve of the eye. In glaucoma, irregularities in the ocular aqueous outflow system cause an elevation in intraocular pressure (IOP) with subsequent death of retinal ganglion cells, resulting in loss of vision. Primary angle closure glaucoma is a major form of blinding disease in Asia and worldwide. Primary angle-closure glaucoma may be caused when the iris is pushed or pulled against the drainage channels at the angle of the anterior chamber of the eye. High resolution visualization of the aqueous outflow system inside the eye would be of great diagnostic value toward understanding disease condition and could allow for monitoring of medical and/or surgical interventions that decrease intraocular pressure as in the case of primary angle-closure glaucoma disease. The aqueous outflow system includes the trabecular meshwork, the Schlemm's canal, and the collector channels. However, none of the currently available clinical imaging techniques such as gonioscopy, Optical Coherence Tomography (OCT), ultrasound biomicroscopy (UBM), anterior segment optical coherence tomography (ASOCT) and EyeCam™ can image the trabecular meshwork with molecular specificity and sufficient spatial resolution of about 1 to 5 pun. While OCT may be clinically effective in measuring the geometrical angle of the iridocorneal angle for indicating angle closure and may achieve imaging with a resolution of several microns, it may not be able to effectively image trabecular meshwork structures, due to the lack of image contrast in OCT images. Alternatively, fluorescence imaging modality may be used to image biological samples because of its ability to specifically image sub-cellular features of interest by attaching fluorescent labels to the region of interest. Wide-field and confocal microscopic imaging techniques may make use of the fluorescence imaging to provide imaging with contrast. However, with such techniques, out-of-focus light may result in blurred images. To overcome the blurring effect, laser point scanning microscopic (LSM) techniques such as confocal and multi-photon techniques may be used. LSM may create images only from in-focus light and may thereby provide intrinsic optical sectioning capabilities. A three dimensional representation of the fluorescent sample may be obtained by digitally uniting a stack of these images. However, in the LSM technique, excitation and collection may occur along the same axis, causing constant irradiation on the entire sample when taking an image stack. The constant irradiation may induce cumulative photodamage within the sample. As such, the currently available LSM techniques may be unsuitable for the purpose of clinical imaging.

### SUMMARY

According to various embodiments, there may be provided an imaging device including a Bessel beam generator configured to provide a Bessel beam; a scanning mirror configured to scan the Bessel beam across a two-dimensional plane; a scan lens configured to receive the Bessel beam from the scanning mirror, a centre of the scan lens being at least substantially a focal length of the scan lens away from the scanning mirror; an illumination tube lens configured to receive the Bessel beam from the scan lens, a centre of the illumination tube lens being at least substantially a sum of the focal length of the scan lens and a focal length of the illumination tube lens away from the centre of the scan lens; an illumination objective lens positioned in direct line-of-sight to a specimen, the illumination objective lens configured to receive the Bessel beam from the illumination tube lens and further configured to illuminate the specimen with the Bessel beam, wherein a centre of the illumination objective lens is at least substantially the focal length of the illumination tube lens away from the centre of the illumination tube lens; and a detection optics arrangement configured to receive a reflected beam from the specimen.

According to various embodiments, there may be provided a method for imaging a specimen, the method including: generating a Bessel beam using a Bessel beam generator; scanning the Bessel beam across a two-dimensional plane using a scanning mirror; receiving the Bessel beam from the scanning mirror using a scan lens, a centre of the scan lens being at least substantially a focal length of the scan lens away from the scanning mirror; receiving the Bessel beam from the scan lens using an illumination tube lens, a centre of the illumination tube lens being at least substantially a sum of the focal length of the scan lens and a focal length of the illumination tube lens away from the centre of the scan lens; receiving the Bessel beam from the illumination tube lens and illuminating the specimen with the Bessel beam using an illumination objective lens, the illumination objective lens being positioned in direct line-of-sight to the specimen, wherein a centre of the illumination objective lens is at least substantially the focal length of the illumination tube lens away from the centre of the illumination tube lens; and receiving a reflected beam from the specimen using a detection optics arrangement.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1A shows a conceptual diagram of an imaging device according to various embodiments.

FIG. 1B shows an imaging device according to various embodiments.

FIG. 2 shows a flow diagram of a method for imaging a specimen according to various embodiments.

FIG. 3 shows a diagram illustrating the aqueous outflow system of a normally functioning eye.

FIG. 4 shows a diagram illustrating the concept of gonioscopy.

FIG. 5 shows a schematic diagram illustrating the imaging geometry of an imaging device according to various embodiments.

FIG. 6 shows a Bessel beam generator according to various embodiments.

FIG. 7A shows a schematic diagram illustrating the effect of an opaque obstacle on an imaging Gaussian light beam.

FIG. 7B shows a schematic diagram illustrating the effect of an opaque obstacle on an imaging Bessel light beam.

FIG. 8 shows the lens arrangement of an imaging device according to various embodiments.

FIG. 9 shows a diagram illustrating the scanning optics configuration that may be employed in an imaging device, according to various embodiments.

FIG. 10 shows an imaging device according to various embodiments.

FIG. 11 shows an imaging device according to various embodiments.

FIG. 12 shows an imaging device according to various embodiments.

FIG. 13 shows a schematic diagram of a prototype of an imaging device according to various embodiments.

FIG. 14 shows a series of images obtained by illuminating a specimen with a Gaussian laser beam using an imaging device according to various embodiments.

FIG. 15 shows a series of images obtained by illuminating a specimen with a Bessel beam using an imaging device according to various embodiments, wherein the specimen has no obstacles.

FIG. 16 shows a series of images obtained by illuminating a specimen with a Bessel beam using an imaging device according to various embodiments.

FIG. 17 shows a graph showing the background level of fluorescence count in the untreated eye of the New Zealand white rabbit.

FIG. 18 shows a graph showing the fluorescence count in the eye of the rabbit in which fluorescein is applied.

FIG. 19 shows an image showing a representative image obtained using an imaging device according to various embodiments.

FIG. 20 shows a series of images of the angle region, obtained at different depths.

FIG. 21 shows eye images obtained using an imaging device according to various embodiments.

FIG. 22 shows a series of snapshots of a porcine cornea.

FIG. 23 shows a series of porcine corneal images at different depths.

FIG. 24 shows a diagram showing a grid drawn on the laser marking software and the direction of scan.

FIG. 25A shows an image of a New Zealand white rabbit's healthy cornea.

FIG. 25B shows an image of the white rabbit's cornea 10 days after the infection.

FIG. 26 shows a graph showing how a fluorescent intensity on the corneal surface of an eye varies with a Bessel beam wavelength.

FIG. 27 shows a graph showing the fluorescent level in the anterior chamber of the eye of FIG. 26.

### DESCRIPTION

Embodiments described below in context of the devices are analogously valid for the respective methods, and vice versa. Furthermore, it will be understood that the embodiments described below may be combined, for example, a part of one embodiment may be combined with a part of another embodiment.

In this context, the imaging device as described in this description may include a memory which is for example used in the processing carried out in the imaging device. A memory used in the embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In the specification the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

In order that the invention may be readily understood and put into practical effect, particular embodiments will now be described by way of examples and not limitations, and with reference to the figures.

Various embodiments are provided for devices, and various embodiments are provided for methods. It will be understood that basic properties of the devices also hold for the methods and vice versa. Therefore, for sake of brevity, duplicate description of such properties may be omitted.

It will be understood that any property described herein for a specific device may also hold for any device described herein. It will be understood that any property described herein for a specific method may also hold for any method described herein. Furthermore, it will be understood that for any device or method described herein, not necessarily all the components or steps described must be enclosed in the device or method, but only some (but not all) components or steps may be enclosed.

The term “coupled” (or “connected”) herein may be understood as optically coupled, electrically coupled or as mechanically coupled, for example attached or fixed, or just in contact without any fixation, and it will be understood that both direct coupling or indirect coupling (in other words: coupling without direct contact) may be provided.

Glaucoma is an eye disease that may result in the loss of sight by damaging the optic nerve of the eye. In glaucoma, irregularities in the ocular aqueous outflow system cause an elevation in intraocular pressure (IOP) with subsequent death of retinal ganglion cells, resulting in loss of vision. Primary angle closure glaucoma is a major form of blinding disease in Asia and worldwide. Primary angle-closure glaucoma may be caused when the iris is pushed or pulled against the drainage channels at the angle of the anterior chamber of the eye. High resolution visualization of the aqueous outflow system inside the eye would be of great diagnostic value toward understanding disease condition and could allow for monitoring of medical and/or surgical interventions that decrease intraocular pressure as in the case of primary angle-closure glaucoma disease. The aqueous outflow system includes the trabecular meshwork, the Schlemm's canal, and the collector channels. However, none of the currently available clinical imaging techniques such as gonioscopy, Optical Coherence Tomography (OCT), ultrasound biomicroscopy (UBM), anterior segment optical coherence tomography (ASOCT) and EyeCam™ can image the trabecular meshwork with molecular specificity and sufficient spatial resolution of about 1 to 5 μm. While OCT may be clinically effective in measuring the geometrical angle of the iridocorneal angle for indicating angle closure and may achieve imaging with a resolution of several microns, it may not be able to effectively image trabecular meshwork structures, due to the lack of image contrast in OCT images. Alternatively, fluorescence imaging modality may be used to image biological samples because of its ability to specifically image sub-cellular features of interest by attaching fluorescent labels to the region of interest. Wide-field and confocal microscopic imaging techniques may make use of the fluorescence imaging to provide imaging with contrast. However, with such techniques, out-of-focus light may result in blurred images. To overcome the blurring effect, laser point scanning microscopic (LSM) techniques such as confocal and multi-photon techniques may be used. LSM may create images only from in-focus light and may thereby provide intrinsic optical sectioning capabilities. A three-dimensional representation of the fluorescent sample may be obtained by digitally uniting a stack of these images. However, in the LSM technique, excitation and collection may occur along the same axis, causing constant irradiation on the entire sample when taking an image stack. The constant irradiation may induce cumulative photodamage within the sample. As such, the currently available LSM techniques may not be suitable for the purpose of clinical imaging.

In the context of various embodiments, the phrase “selective plane illumination microscopy” may be but is not limited to being interchangeably referred to as a “SPIM” or light sheet microscopy.

In the context of various embodiments, the phrase “scan lens” may be but is not limited to being interchangeably referred to as a “visible scan lens”.

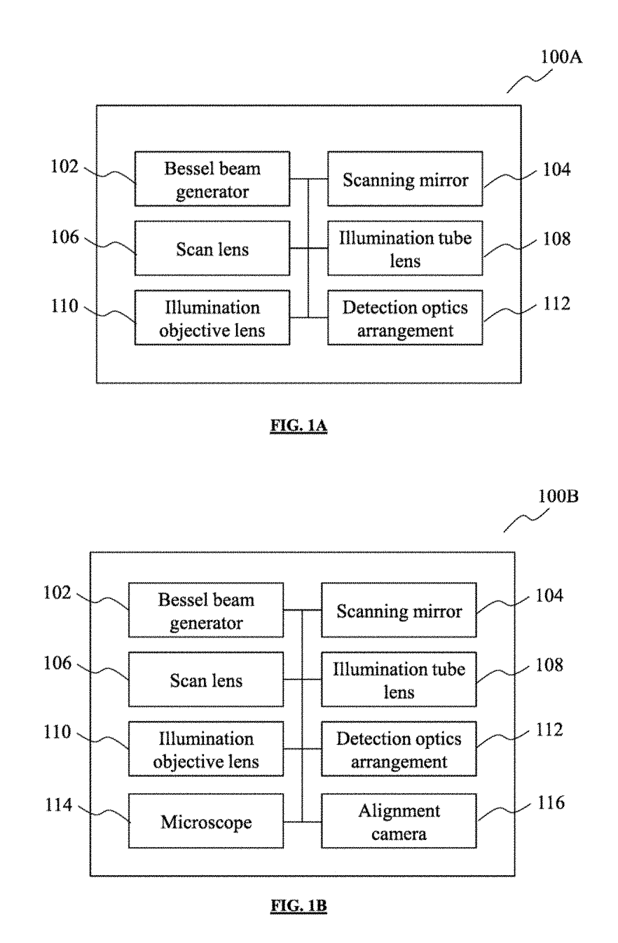

FIG. 1A shows a conceptual diagram of an imaging device 100A according to various embodiments. The imaging device 100A may include a Bessel beam generator 102 configured to provide a Bessel beam; a scanning mirror 104 configured to scan the Bessel beam across a two-dimensional plane; a scan lens 106 configured to receive the Bessel beam from the scanning mirror 104, a centre of the scan lens 106 being at least substantially a focal length of the scan lens 106 away from the scanning mirror 104; an illumination tube lens 108 configured to receive the Bessel beam from the scan lens 106, a centre of the illumination tube lens 108 being at least substantially a sum of the focal length of the scan lens 106 and a focal length of the illumination tube lens 108 away from the centre of the scan lens 106; an illumination objective lens 110 positioned in direct line-of-sight to a specimen, the illumination objective lens 110 configured to receive the Bessel beam from the illumination tube lens 108 and further configured to illuminate the specimen with the Bessel beam, wherein a centre of the illumination objective lens 110 is at least substantially the focal length of the illumination tube lens 108 away from the centre of the illumination tube lens 108; and a detection optics arrangement 112 configured to receive a reflected beam (e.g. an emitted fluorescence beam) from the specimen.

In other words, according to various embodiments, the imaging device 100A may include a Bessel beam generator 102, a scanning mirror 104, a scan lens 106, an illumination tube lens 108, an illumination objective lens 110 and a detection optics arrangement 112. The Bessel beam generator 102 may be configured to provide a Bessel beam. The scanning mirror 104 may be configured to receive the Bessel beam from the Bessel beam generator 102 and may be further configured to scan the Bessel beam across a two-dimensional plane. The scan lens 106 may be configured to receive the Bessel beam from the scanning mirror 104. The scan lens 106 may be arranged such that a centre of the scan lens 106 is a first distance away from the scanning mirror 104. The first distance may be at least substantially equal to a focal length of the scan lens 106. The illumination tube lens 108 may be configured to receive the Bessel beam from the scan lens 106. The illumination tube lens 108 may be arranged such that a centre of the illumination tube lens 108 is a second distance away from the centre of the scan lens 106. The second distance may be at least substantially equal to a sum of the focal length of the scan lens 106 and a focal length of the illumination tube lens 108. The illumination objective lens 110 may be positioned in direct line-of-sight to a specimen that is to be imaged. The illumination objective lens 110 may be configured to receive the Bessel beam from the illumination tube lens 108 and may be further configured to illuminate the specimen with the Bessel beam. The illumination objective lens 110 may be arranged such that a centre of the illumination objective lens 110 is a third distance away from the centre of the illumination tube lens 108. The third distance may be at least substantially equal to the focal length of the illumination tube lens 108.

According to various embodiments, the reflected beam may be a reflection of the Bessel beam, by the specimen.

According to various embodiments, the reflected beam may be an emitted fluorescence beam. The reflected beam may be a fluorescence beam emitted by fluorescein that is applied to the specimen, as a result of the specimen receiving the Bessel beam from the imaging device.

The Bessel beam generator 102 may include a laser generator configured to generate a Gaussian beam; a collimator coupled to the laser generator; an aperture; and an axicon lens. The collimator, for example, a fiber collimator, may be configured to receive the Gaussian beam and may be further configured to collimate the Gaussian beam. The aperture may be configured to receive the Gaussian beam from the collimator and may be further configured to pass through a further collimated Gaussian beam. The aperture may be variable for adjusting a depth of focus of the imaging device 100. The axicon lens may have an apex angle at least substantially in a range of 168° to 178° and may be configured to convert the further collimated Gaussian beam into a Bessel beam. The Bessel beam generator may further include a single mode fiber for coupling the laser generator to the collimator, and may further include a further collimator, such a collimation lens configured to collimate the Bessel beam. The collimator or the further collimator may have a numerical aperture at least substantially equal to 0.26. The collimator or the further collimator may have a focal length at least substantially equal to 34.74 mm.

The detection optics arrangement 112 may include a first arrangement including a detection objective lens, a detection tube lens and an imaging sensor. The detection objective lens may be positioned at least substantially orthogonal to the illumination objective lens. The detection objective lens may be configured to receive the reflected beam from the specimen. The detection tube lens may be coupled to a back aperture of the detection objective lens for receiving the reflected beam from the detection objective lens. The imaging sensor may be configured to receive the reflected beam from the detection tube lens. The first arrangement may further include a notch filter positioned between the detection objective lens and the detection tube lens. The detection objective lens may be at least substantially similar to the illumination objective lens while the detection tube lens may be at least substantially similar to the illumination tube lens. The imaging device may have a lateral resolution determinable based on a numerical aperture (NA) of the detection objective lens. The lateral resolution may be inversely proportional to the NA of the detection objective lens. The imaging device may be used to image a specimen overlaid with a fluorophore, for example, fluorescein. The Bessel beam generator 102 may be configured to generate a Bessel beam having a wavelength at least substantially corresponding to an excitation wavelength of the fluorophore. The lateral resolution may be further determinable based on an emission wavelength of the fluorophore. The lateral resolution may be proportional to the emission wavelength of the fluorophore.

The detection optics arrangement 112 may include a second arrangement including a beam splitter, a focusing lens and an imaging sensor. The second arrangement may be a replacement for the first arrangement. The detection optics arrangement 112 may also include both the first arrangement and the second arrangement. The beam splitter may be positioned between the illumination tube lens and the illumination objective lens. The beam splitter may be configured to receive the reflected beam from the specimen through the illumination objective lens and may be further configured to partially reflect the reflected beam in a direction at least substantially orthogonal to the reflected beam. The focusing lens may be configured to receive the partial reflection of the reflected beam from the beam splitter. The imaging sensor may be configured to receive the partial reflection of the reflected beam from the focusing lens. The second arrangement may further include a variable neutral density filter positioned between the focusing lens and the imaging sensor.

FIG. 1B shows an imaging device 100B according to various embodiments. The imaging device 100B may be similar to the imaging device 100A, in that it may include a Bessel beam generator 102, a scanning mirror 104, a scan lens 106, an illumination tube lens 108, an illumination objective lens 110 and a detection optics arrangement 112. The imaging device 100B may further include a microscope 114. The microscope 114 may be configured to provide optical magnification. The microscope 114 may be a mini USB digital microscope. The imaging device 100B may also include an alignment camera 116. The alignment camera 116 may be incorporated with white light illumination. The alignment camera 116 may be configured for a user of the imaging device to check an initial positioning and alignment of the specimen.

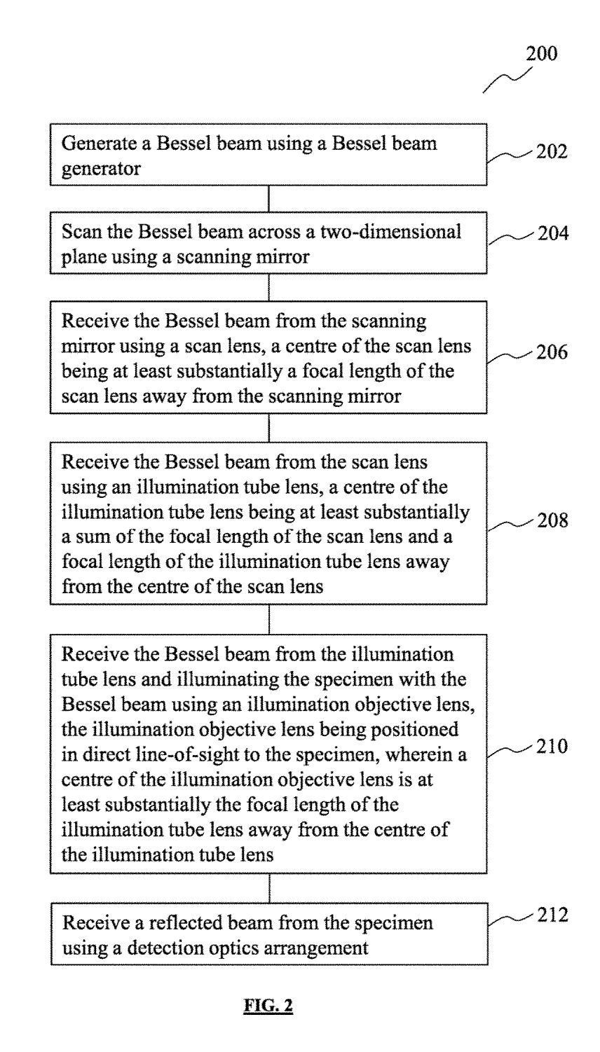

FIG. 2 shows a flow diagram 200 of a method for imaging a specimen, according to various embodiments. The method may include 202, in which a Bessel beam is generated using a beam generator; 204, in which the Bessel beam is scanned across a two-dimensional plane using a scanning mirror; 206, in which the Bessel beam is received from the scanning mirror using a scan lens, a centre of the scan lens being at least substantially a focal length of the scan lens away from the scanning mirror; 208, in which the Bessel beam is received from the scan lens using an illumination tube lens, a centre of the illumination tube lens being at least substantially a sum of the focal length of the scan lens and a focal length of the illumination tube lens away from the centre of the scan lens; 210, in which the Bessel beam is received from the illumination tube lens and the specimen is illuminated with the Bessel beam using an illumination objective lens, the illumination objective lens being positioned in direct line-of-sight to the specimen, wherein a centre of the illumination objective lens is at least substantially the focal length of the illumination tube lens away from the centre of the illumination tube lens; and 212, in which a reflected beam from the specimen is received using a detection optics arrangement.

Glaucoma refers to a group of eye conditions that damages the optic nerve and may thereby result in loss of sight. The angle-closure glaucoma, also known as the closed-angle glaucoma, or narrow angle glaucoma, or primary angle closure glaucoma, or acute glaucoma, is one type of glaucoma. The angle-closure glaucoma is a major form of blinding disease in Asia and worldwide. It may be caused by a blockage of the outflow of the aqueous humour from within the eye.

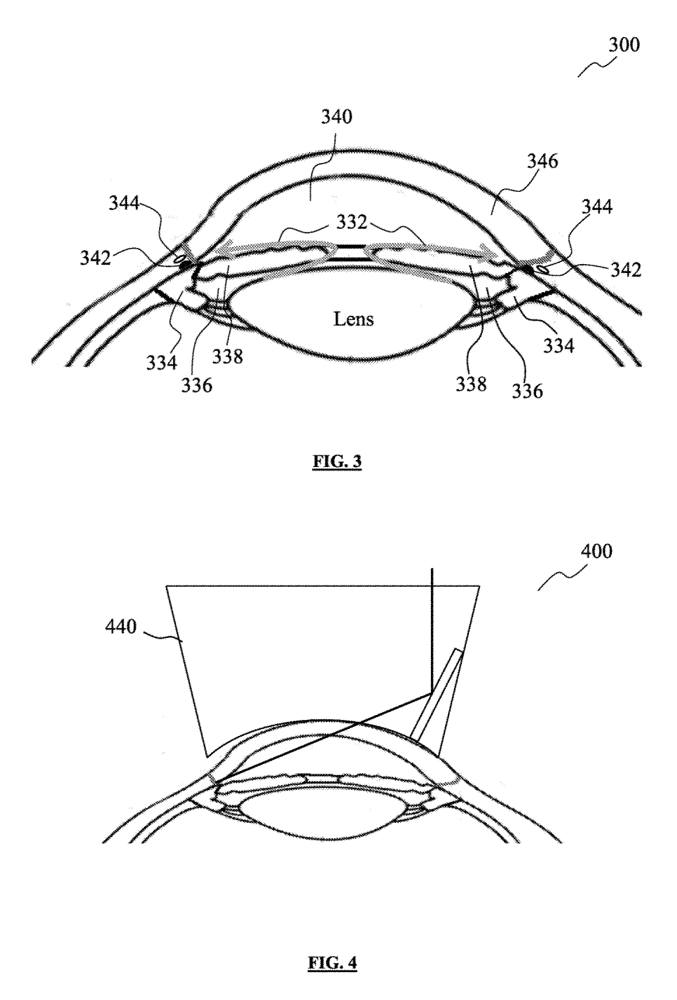

FIG. 3 shows a diagram 300 illustrating the aqueous outflow system of a normally functioning eye. The diagram 300 shows the direction of outflow of the aqueous humour 332. The aqueous humour 332, may also be referred herein as an aqueous fluid, flows from the ciliary body 334 to the posterior chamber 336, and then flows through the pupil of the iris 338 into the anterior chamber 340. The trabecular meshwork 342 then drains the aqueous humour 332 out of the eye, via the Schlemm's canal 344 and the Collectors channel. In an eye suffering from angle-closure glaucoma, the iris 338 is pushed or pulled against the cornea 346, obstructing the outflow of the aqueous humour 332 from the posterior chamber 336 into the anterior chamber 340 and then out of the trabecular meshwork 342. In other words, the iridocorneal angle is completely closed, thereby closing off the drainage of the aqueous humour 332 out of the eye. As a result, the aqueous humour 332 accumulates within the eye, causing an elevation in intraocular pressure (IOP). The elevation in the IOP causes retinal ganglion cells to die, resulting in a loss of vision. As glaucoma causes the trabecular meshwork 342 to degenerate, characterizing the cell and collagen structures in the trabecular meshwork 342 may allow early diagnosis, disease monitoring, as well as fundamental studies of the glaucoma disease mechanism. High resolution visualization of the aqueous outflow system would be of great diagnostic value toward understanding the glaucoma disease condition and may allow for the monitoring of medical and/or surgical interventions that may decrease the IOP. However, the anterior chamber angle (ACA) is not easily visible with direct external observation, as the oblique angle required for viewing the ACA exceeds the critical angle of the cornea. In other words, light illuminating the anterior chamber 340 at the oblique angle will be completely reflected backed into the eye when the light hits the surface of the cornea 346.

FIG. 4 shows a diagram 400 illustrating the concept of gonioscopy. Gonioscopy is the current reference standard for assessing the ACA. In gonioscopy, a goniolens 440 is used in conjunction with a slit lamp or an operating microscope to gain a view of the iridocorneal angle. The goniolens 440 may be placed directly on the cornea. Gonioscopy offers the advantage of being able to visualize a whole quadrant of the ACA at one time. The numerical aperture of the singlet lens is about 0.2, yielding a theoretical lateral resolution of about 2 μm However, gonioscopy also has its disadvantages. Gonioscopy is a contact method which requires contacting of the patient's cornea and the contacting of the cornea may be painful to the patient. Gonioscopy also requires bulky instrumentation devices. The imaging obtained using gonioscopy may also be very difficult to translate to a clinical standard.

According to various embodiments, the imaging device may be capable of three-dimensional volume imaging of the aqueous outflow system of eyes with relatively good resolution. The imaging device may employ a scanning optical system that scans with Bessel beams, in other words, perform Bessel beam light sheet microscopy. The imaging device may form a Bessel beam by illuminating an axicon with a Gaussian beam output of a laser. The imaging device may include an excitation arm, the excitation arm including collimation optics and scanning optics. The scanning optics may include a galvo mirror, a scan lens and a tube lens arranged in 4-f configuration along with an excitation objective lens. The imaging device may further include a detection optics arrangement. There may be two types of detection optics arrangements, namely a corneal imaging detection optics arrangement; and an angle imaging detection optics arrangement. The corneal imaging detection optics arrangement may include an epi-illumination configuration. The angle imaging detection optics arrangement may include an orthogonal detection configuration. The imaging device may further include an alignment camera incorporated with white light illumination. The alignment camera may be employed for checking the initial positioning and alignment of the eye in order to have the right illumination at the desired area. The image contrast and anatomical discrimination in the optical slices obtained using the Bessel beam imaging may be improved, by overlaying the region of the eye with a fluorescent substance, such as fluorescein, which may be an ideal fluorescent sample for ocular imaging. The imaging device may further include fluorescent filters, for example, dichroic filters or notch filters, for angle imaging in the detection optics arrangement, also referred herein as the collection arm.

According to various embodiments, a method for imaging specimens may include using selective plane illumination microscopy (SPIM) techniques. SPIM may also be known as light sheet microscopy. In existing SPIM technologies, a static sheet of excitation light, also referred herein as a light sheet, may be produced onto a sample plane by focusing a Gaussian beam through a cylindrical lens. The fluorescence light emerging from the sample plane may be collected through a microscope collection objective lens. The collection objective lens may be placed along the axis orthogonal to the light sheet. The resolution along the axial direction may be determined by the thickness of the light sheet, while the resolution along the transverse direction may be determined by the numerical aperture of the collection objective lens. However, the light sheet produced as such may have the disadvantages of being broadened deep inside the sample, due to scattering and aberrations. Also, in order to achieve a large field of view, the depth of field of the cylindrical lens may need to be large and this large depth of field may be achieved by using low numerical aperture lenses. Using low numerical aperture lenses has the disadvantage of reducing the optical sectioning ability, as the thickness of the generated cylindrical beam is increased. A method according to various embodiments may realise the light sheet by scanning a focused beam in one direction. A digitally scanned light sheet may be generated by rapidly scanning a Bessel beam up and down. The method according to various embodiments may offer benefits over the existing SPIM methods, such as improved axial resolution, reduction in light scattering artifacts as well as increased penetration depth. The method may provide the capability to perform 2D optical sectioning in large fields of view. This method also reduces the photodamage caused to the sample, as compared to existing techniques, as the irradiation of the sample is restricted to the plane under observation. This method may achieve high acquisition speed and low exposure of the sample to illumination light. Additionally, the longitudinal extent of the light-sheet may be set independent of its central thickness. The self-reconstruction property of the Bessel-beam may be ideal for imaging through an inhomogeneous medium. The low photo-bleaching, low photodamage and high acquisition speed of the method makes it ideal for in vivo ocular imaging. The method may penetrate the sclera to image a region of the conventional outflow system. The confined excitation provides optical sectioning automatically by producing minimal out-of-focus fluorescence background.

According to various embodiments, a method for imaging specimens may exploit the properties of Bessel beam in a fluorescence overlay ambience. A digitally scanned light sheet may be generated by rapidly scanning a Bessel beam up and down. A static light sheet composed of Bessel beams may be generated with a combination of cylindrical optics and objective lenses. The self-reconstruction property of the Bessel beam may reduce the shadowing and scattering artifacts in plane illumination microscopy. The scanned Bessel beam may generate a much thinner light sheet, resulting in better axial resolution. The confined excitation may provide optical sectioning automatically by producing minimal out-of-focus fluorescence background.

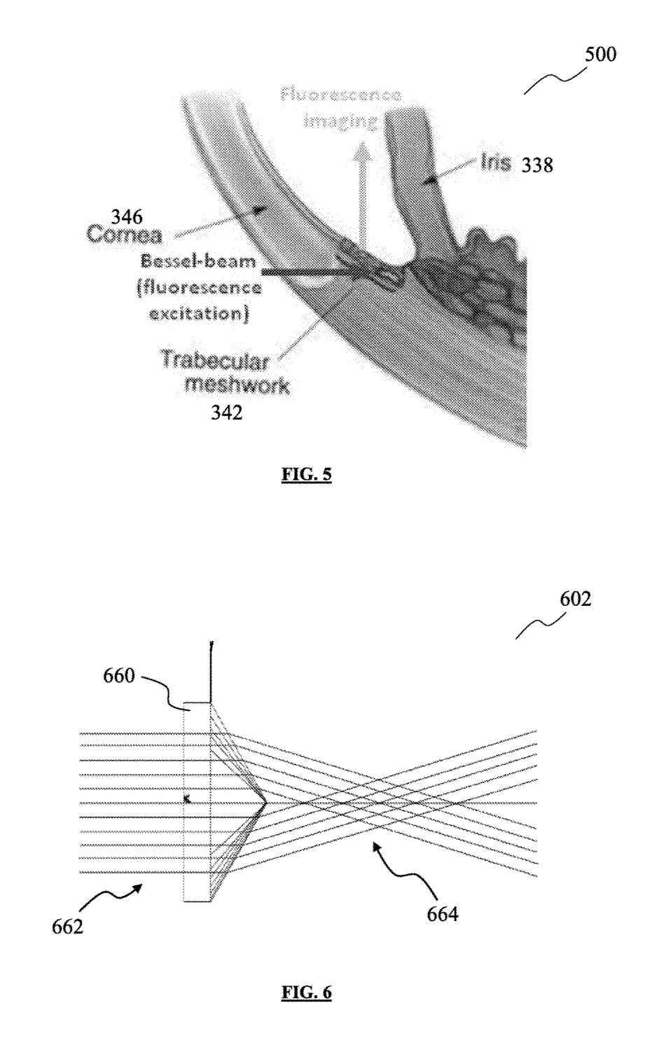

FIG. 5 shows a schematic diagram 500 illustrating the imaging geometry of an imaging device according to various embodiments. A patient who will be undergoing an eye-imaging procedure may have a fluorophore substance, such as fluorescein, applied on his or her eye. The imaging device may illuminate the trabecular meshwork 342 of the eye with the Bessel beam. The Bessel beam may be reflected off the trabecular meshwork 342, to be received by the imaging device. The Bessel beam provided by the imaging device may have a wavelength at least substantially corresponding to an excitation wavelength of the fluorophore, such that the reflected beam is fluorescent. The fluorescence reflection may enhance contrast in the image received by the imaging device, such that the details of the trabecular meshwork 342 may be more easily distinguished from the image obtained.

FIG. 6 shows a Bessel beam generator 602 according to various embodiments. The Bessel beam generator 602 may include an axicon lens 660. The axicon lens 660 may refract an input light 662 to provide a Bessel beam 664. The Bessel beam generator 602 may be similar to, or identical to, the Bessel beam generator 102 of FIGS. 1A and 1B. The Bessel beam generator 602 may be configured to generate a Bessel beam.

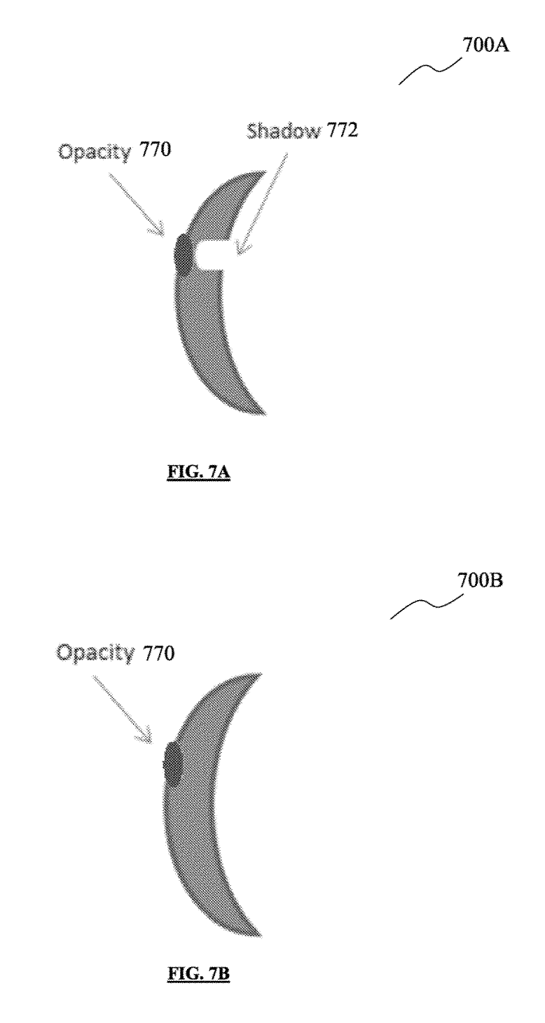

FIG. 7A shows a schematic diagram 700A illustrating the effect of an opaque obstacle on an imaging Gaussian light beam. The opaque obstacle, herein referred to as opacity 770, may result in a shadow 772.

FIG. 7B shows a schematic diagram 700B illustrating the effect of an opaque obstacle on an imaging Bessel light beam. A Bessel beam has the property of being non-diffractive, in other words, as the Bessel beam propagates, the Bessel beam does not spread out. A Bessel beam also has the unique property of being self-healing, in that the Bessel beam can re-form a point further down the beam axis, even if the beam is partially obstructed at one point along the beam axis. Therefore, as illustrated in the schematic diagram 700B, the Bessel beam may reconstruct around the opacity 770 and as a result, no shadow is formed.

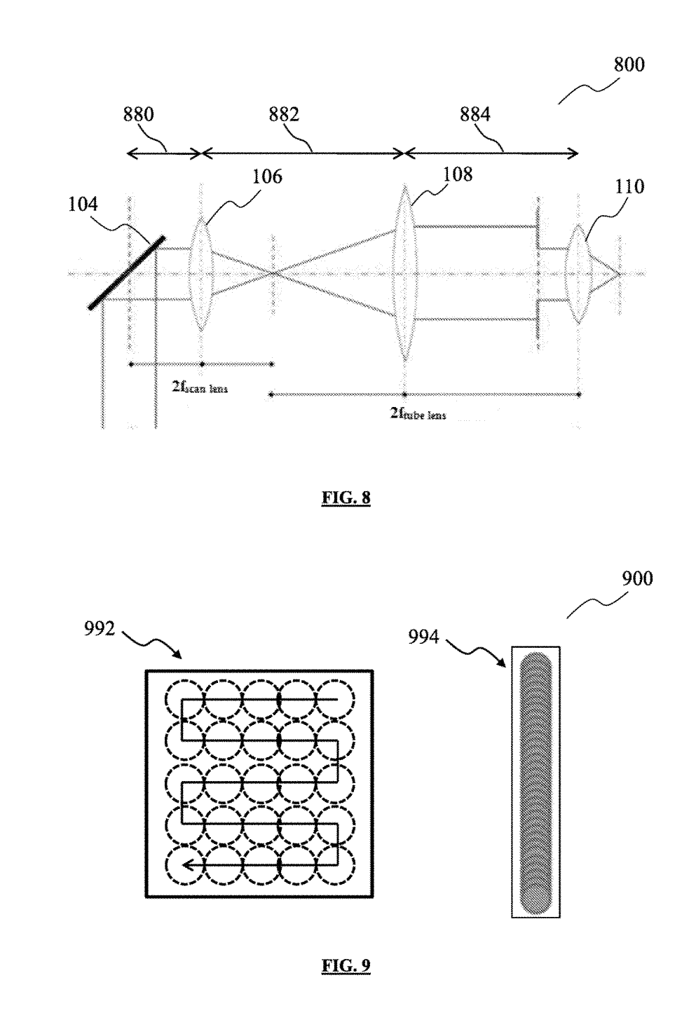

FIG. 8 shows the lens arrangement 800 of an imaging device according to various embodiments. The imaging device may be any one of the imaging devices 100A or 100B. The scan lens 106 and the illumination tube lens 108 may be arranged after the scanning mirror 104 in a “4F” configuration. The focal length of the scan lens 106 may be referred herein as f_{scan lens}. The focal length of the illumination tube lens 108 may be referred herein as f_{tube lens}. A centre of the scan lens 106 may be at least substantially f_{scan lens} away from the scanning mirror 104. A centre of the illumination tube lens 108 may be at least substantially a sum of f_{scan lens} and f_{tube lens} away from the centre of the scan lens 106. A centre of the illumination objective lens 110 may be at least substantially f_{tube lens} away from the centre of the illumination tube lens 108. In other words, the scan lens 106 is positioned at a first distance 880 away from the centre of the scanning mirror 104. The first distance 880 may be at least substantially equal to f_{scan lens}. The illumination tube lens 108 is positioned at a second distance 882 away from the centre of the scan lens 106. The second distance 882 may be at least substantially equal to f_{scan lens}+f_{tube lens}. The illumination objective lens 110 may be positioned at a third distance 884 away from the centre of the illumination tube lens 108. The third distance 884 may be at least substantially equal to the f_{tube lens}.

FIG. 9 shows a diagram 900 illustrating the scanning optics configuration that may be employed in an imaging device, according to various embodiments. The scanning mirror of the imaging device may be configured to scan a received beam across a two-dimensional plane, in a scanning pattern indicated by the arrow in the sub-diagram 992. The scanning pattern may be a raster scanning pattern, in other words, the scanning may be performed line-by-line. The scanning speed of the scanning mirror, as well as the scanning area of the scanning mirror, may be configurable by software. The sub-diagram 994 shows the light sheet formed by the digital raster scanning shown in the sub-diagram 992.

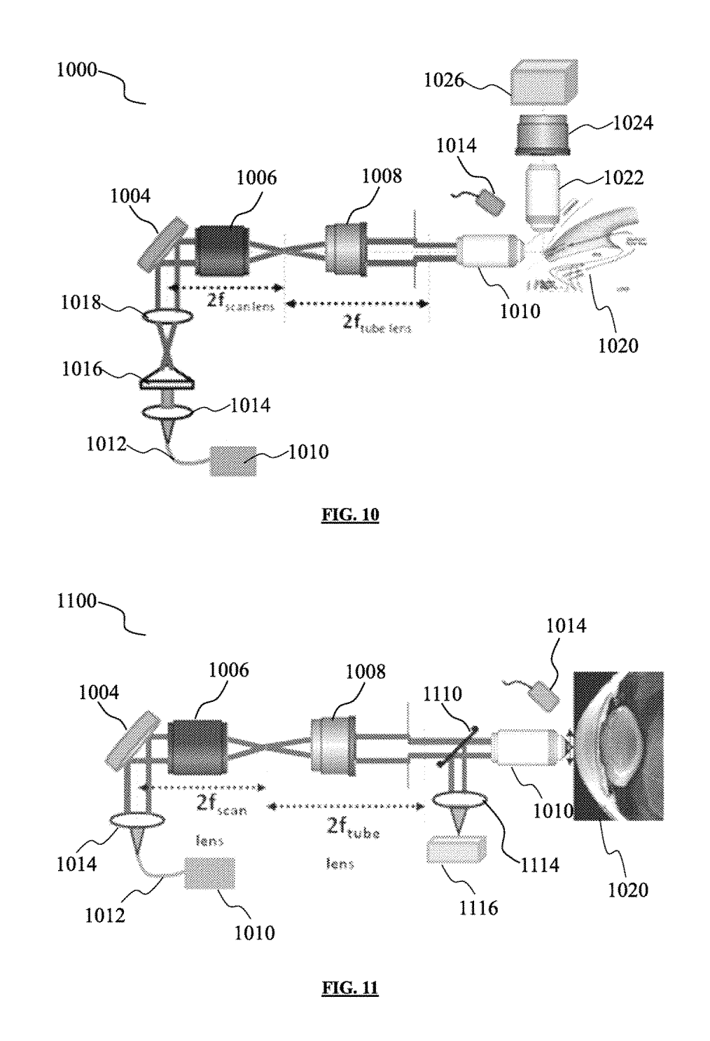

FIG. 10 shows an imaging device 1000 according to various embodiments. The imaging device 1000 may include a Bessel beam generator, a scanning mirror 1004, a scan lens 1006, an illumination tube lens 1008, an illumination objective lens 1010 and a detection optics arrangement. The Bessel beam generator may include a laser generator 1010, a collimator, and an axicon lens 1016. The collimator may include a single mode fiber 1012 and may further include a fiber collimator 1014. The laser generator 1010 may be configured to provide a Gaussian beam. The single mode fiber 1012 may be configured to receive the Gaussian beam from the laser generator 1010 and further configured to relay the Gaussian beam to the fiber collimator 1014. The fiber collimator 1014 may be configured to collimate the Gaussian beam and provide the collimated beam to the axicon lens 1016. The axicon lens 1016 may be configured to receive the further collimated Gaussian beam and further configured to output a Bessel beam. The Bessel beam generator may further include a collimation lens 1018 configured to collimate the Bessel beam. The scanning mirror 1004 may be configured to receive the Bessel beam from the collimation lens 1018 and further configured to scan the Bessel beam across a two-dimensional plane. The scan lens 1006 may be configured to receive the Bessel beam from the scanning mirror 1004. The illumination tube lens 1008 may be optically coupled to the scan lens 1006 and may be configured to receive the Bessel beam from the scan lens 1006. The illumination objective lens 1010 may be optically coupled to the illumination tube lens 1008, to receive the Bessel beam from the illumination tube lens 1008. The illumination objective lens 1010 may be positioned in direct line-of-sight to a specimen 1020, for example, an eye, to illuminate the specimen 1020 with the Bessel beam. The Bessel beam may be reflected from the specimen, the reflection referred herein as the reflected beam. The reflected beam may be a reflection of the Bessel beam, by the specimen. The detection optics arrangement may be configured to receive the reflected beam from the specimen 1020. The detection optics arrangement may include a detection objective lens 1022, a detection tube lens 1024 and an imaging sensor 1026. The detection objective lens 1022 may be positioned at least substantially orthogonal to the illumination objective lens 1010, and may be configured to receive the reflected beam from the specimen 1020. The detection tube lens 1024 may be coupled to a back aperture of the detection objective lens 1022 and may be configured to receive the reflected beam from the detection objective lens 1022. The imaging sensor 1026 may be a low light sensitive Complementary Metal-Oxide-Semiconductor (CMOS) sensor. Alternatively, the imaging sensor 1026 may be a scientific Complementary Metal-Oxide-Semiconductor (sCMOS) camera. The imaging device 1000 may further include a microscope 1014. The microscope 1014 may be a mini USB digital microscope.

The imaging device 1000 may be configured to scan a focused Bessel beam to create a light sheet for imaging the aqueous outflow system inside an eye. The imaging device may be capable of three-dimensional volume imaging of the aqueous outflow system of the eye with a relatively high resolution. The imaging device 1000 may be used to perform angle imaging inside the eye. The imaging device 1000 may be able to provide a 360° view of the angle region in the eye and may provide images wherein the peripheral anterior structures of the eye are discernable. The imaging device may be configured to form the Bessel beam by illuminating an axicon with a Gaussian beam output of a laser. The imaging device 1000 may include an excitation arm and a collection arm. The excitation arm may include collimation optics and scanning optics. The scanning optics may include a galvo mirror, the scan lens 1006 and a tube lens arranged in 4-f configuration. The galvo mirror may be referred herein as the scanning mirror 1004. The tube lens may be referred herein as the illumination tube lens 1008. The scanning optics may further include an excitation objective lens, herein referred to as the illumination objective lens 1010. The collection arm may be referred herein as the detection optics arrangement. The collection arm may include a collection objective lens positioned orthogonal to the excitation objective lens. The collection objective lens may be referred herein as the detection objective lens 1022. The collection arm may further include a dichroic filter, a tube lens and a CMOS sensor. The CMOS sensor may be an embodiment of the imaging sensor 1026. The tube lens may be an embodiment of the focusing lens 1024. The self-reconstructing property of the Bessel beams generated by the axicon lens 1016 may increase the image contrast, as well as reduce scattering and shadow artifacts in the images of the trabecular meshwork region inside the eye. The image contrast and anatomical discrimination in the optical slices obtained using the Bessel beam imaging may be improved by overlaying the trabecular meshwork region with fluorescein.

FIG. 11 shows an imaging device 1100 according to various embodiments. The imaging device 1100 may include a laser generator 1010, a single mode fiber 1012 and a fiber collimator 1014, a scanning mirror 1004, a scan lens 1006, an illumination tube lens 1008, an illumination objective lens 1010 and a detection optics arrangement. The laser generator 1010 may be configured to provide a laser beam. The laser beam may be a Gaussian beam. The single mode fiber 1012 may be configured to receive the Gaussian beam from the laser generator 1010 and may be further configured to relay the Gaussian beam to the fiber collimator 1014. The fiber collimator 1014 may be configured to collimate the Gaussian beam and provide the collimated beam to the scanning mirror 1004. The scanning mirror 1004 may be configured to receive the collimated beam and further configured to scan the collimated beam across a two-dimensional plane. The scan lens 1006 may be configured to receive the beam from the scanning mirror 1004. The illumination tube lens 1008 may be optically coupled to the scan lens 1006 and may be configured to receive the beam from the scan lens 1006. The illumination objective lens 1010 may be optically coupled to the illumination tube lens 1008, to receive the beam from the illumination tube lens 1008. The illumination objective lens 1010 may be positioned in direct line-of-sight to a specimen 1020, for example, an eye, to illuminate the specimen 1020 with the beam. The reflection of the beam off the specimen 1020 may be referred herein as a reflected beam. The detection optics arrangement may include a beam splitter 1110, a focusing lens 1114 and an imaging sensor 1116. The beam splitter 1110 may be positioned between the illumination tube lens 1008 and the illumination objective lens 1010. The beam splitter 1110 may be configured to receive the reflected beam from the specimen 1020 through the illumination objective lens 1010 and may be further configured to partially reflect the reflected beam in a direction at least substantially orthogonal to the reflected beam. The beam splitter 1110 may be a pellicle beam splitter. The beam splitter may have a reflection to transmission ratio at least substantially equal to 45:55. The focusing lens 1114 may be configured to receive the partial reflection of the reflected beam from the beam splitter 1110. The focusing lens 1114 may be a tube lens. The imaging sensor 1116 may be configured to receive the partial reflection of the reflected beam from the focusing lens 1114. The imaging sensor 1116 may be a charge-coupled device (CCD) camera. The CCD may have a frame rate of more than 15 frames per second.

The imaging device 1100 may be similar to the imaging device 1000, except that the imaging device 1100 has a different detection optics arrangement from the imaging device 1000. The imaging device 1100 may be used for corneal imaging. The detection optics arrangement of the imaging device 1100 may employ a reflection-based scanning imaging scheme. The imaging device 1100 may perform imaging on a larger two-dimensional area at the corneal surface using the same galvo-scanning-tube lens combinations as the imaging device 1000. A series of two-dimensional images may be captured for different depths. The series of two-dimensional images may be combined to provide topography details, as well as a three-dimensional reconstruction across the entire thickness of the corneal surface.

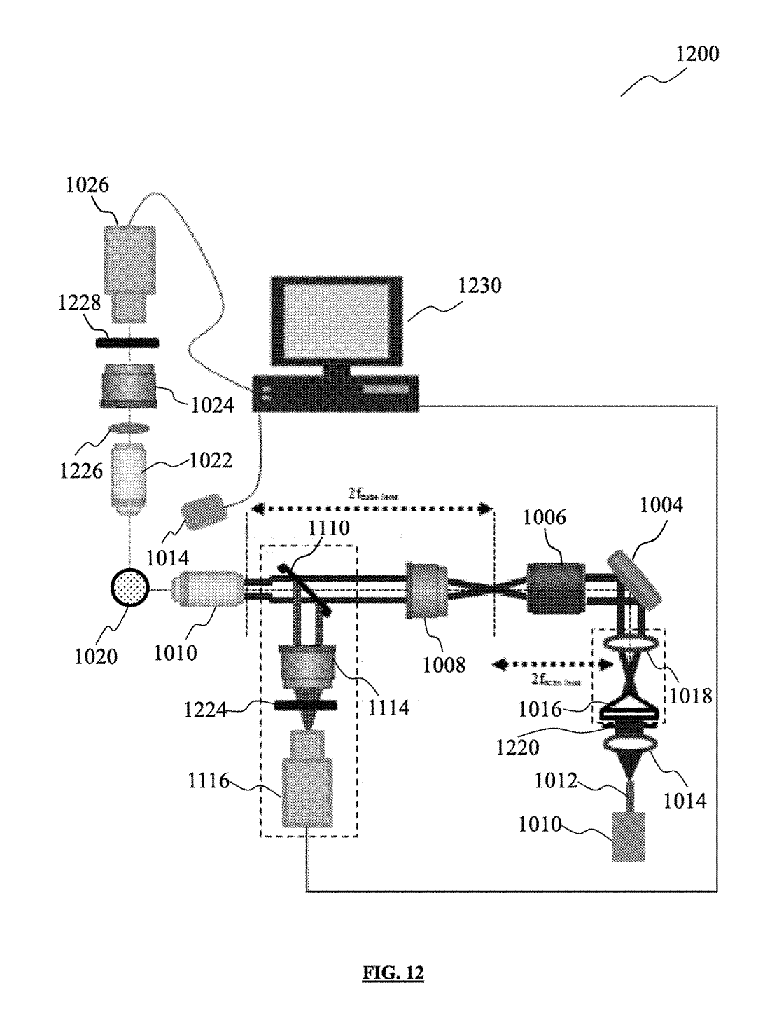

FIG. 12 shows an imaging device 1200 according to various embodiments. The imaging device 1100 may include a Bessel beam generator, a scanning mirror 1004, a scan lens 1006, an illumination tube lens 1008, an illumination objective lens 1010, a first detection optics arrangement and a second detection optics arrangement. The Bessel beam generator may include a laser generator 1010, a collimator, an aperture 1220, and an axicon lens 1016. The collimator may include a single mode fiber 1012 and a fiber collimator 1014. The laser generator 1010 may be configured to provide a Gaussian beam. The single mode fiber 1012 may be configured to receive the Gaussian beam from the laser generator 1010 and further configured to relay the Gaussian beam to the fiber collimator 1014. The fiber collimator 1014 may be configured to collimate the Gaussian beam and provide the collimated beam to the aperture 1220. The aperture 1220 may receive the collimated beam and then pass through a further collimated Gaussian beam. The aperture 1220 may be variable for adjusting a depth of focus of the imaging device 1200. The axicon lens 1016 may be configured to receive the further collimated Gaussian beam and further configured to output a Bessel beam. The Bessel beam generator may further include a collimation lens 1018 configured to collimate the Bessel beam. The scanning mirror 1004 may be configured to receive the Bessel beam from the collimation lens 1018 and further configured to scan the Bessel beam across a two-dimensional plane. The scan lens 1006 may be configured to receive the Bessel beam from the scanning mirror 1004. The illumination tube lens 1008 may be optically coupled to the scan lens 1006 and may be configured to receive the Bessel beam from the scan lens 1006. The illumination objective lens 1010 may be optically coupled to the illumination tube lens 1008, to receive the Bessel beam from the illumination tube lens 1008. The illumination objective lens 1010 may be positioned in direct line-of-sight to a specimen 1020 to illuminate the specimen 1020 with the Bessel beam. The Bessel beam may be reflected from the specimen, the reflection referred herein as the reflected beam. Each of the first detection optics arrangement and the second detection optics arrangement may be configured to receive the reflected beam from the specimen 1020. The first detection optics arrangement may include a detection objective lens 1022, a detection tube lens 1024 and a first imaging sensor 1026. The detection objective lens 1022 may be positioned at least substantially orthogonal to the illumination objective lens 1010, and may be configured to receive the reflected beam from the specimen 1020. The detection tube lens 1024 may be coupled to a back aperture of the detection objective lens 1022 and may be configured to receive the reflected beam from the detection objective lens 1022. The first imaging sensor 1026 may be a low light sensitive Complementary Metal-Oxide-Semiconductor (CMOS) sensor. The first detection optics arrangement may further include a notch filter 1226. The notch filter 1226 may be positioned between the detection objective lens 1022 and the detection tube lens 1024. The first detection optics arrangement may further include a first variable neutral density filter 1228 positioned between the first imaging sensor 1026 and the detection tube lens 1024. The second detection optics arrangement may include a beam splitter 1110, a focusing lens 1114 and a second imaging sensor 1116. The beam splitter 1110 may be positioned between the illumination tube lens 1008 and the illumination objective lens 1010. The beam splitter 1110 may be configured to receive the reflected beam from the specimen 1020 through the illumination objective lens 1010 and may be further configured to partially reflect the reflected beam in a direction at least substantially orthogonal to the reflected beam. The focusing lens 1114 may be configured to receive the partial reflection of the reflected beam from the beam splitter 1110. The imaging sensor 1116 may be configured to receive the partial reflection of the reflected beam from the focusing lens 1114. The imaging sensor 1116 may be a charge-coupled device (CCD) camera. The second detection optics arrangement may further include a second variable neutral density filter 1224 positioned between the focusing lens 1114 and the imaging sensor 1116. The second variable neutral density filter 1224 may alternatively be positioned between the beam splitter 1110 and the focusing lens 1114. The imaging device 1000 may further include a microscope 1014. The imaging device 1200 may be connected to a personal computer 1230, so that the images captured by the imaging sensor 1116 may be processed or analysed subsequently, using the personal computer 1230.

According to various embodiments, the laser generator 1010 may be a compact high performance diode laser configured to provide laser beams having a wavelength of 488 nm or 785 nm. The single mode fiber 1012 may be a 1.5 m long, polarization maintaining fiber which has an alignment free, plug and inter-changeable fiber coupler unit is used for the excitation. The Gaussian beam output of the FC/PC terminated fiber unit, i.e. the single mode fiber 1012, may be collimated using the fiber collimator 1014. The fiber collimator 1014 may provide an illumination to the axicon lens 1016 to generate the Bessel beam. The axicon lens may have an apex angle of at least substantially equal to 176°. The Bessel beam may be collimated and then directed into a scanning mirror 1004. The scanning mirror 1004 may be two-axis galvanometer mirrors. The scan lens 1006 and illumination tube lens 1008 may be placed after the scanning mirror 1004 in a 4F configuration. The illumination tube lens 1008 may be an infinity-corrected tube lens having a focal length of at least substantially equal to 200 mm and a working distance at least substantially equal to 148 mm. The scan lens 1006 may have a focal length of about 70 mm and a working distance of about 54 mm. In the 4F configuration, the scan lens 1006 may be positioned such that the scanning mirror 1004 is at its eye-point while the field aperture plane is at its focal point. Since the illumination objective lens 1010 is infinity-corrected, the illumination tube lens 1008 may be positioned to re-collimate the excitation light. The illumination tube lens 1008 may be paired with a visible scan lens 1006. In this 4F configuration, the illumination tube lens 1008 may relay the scan plane of the laser scanning imaging system to the back aperture of the illumination objective lens 1010. The infinity-corrected long working distance illumination objective lens 1010 may be a plan apochromat lens and may have a magnification of about 20×, a NA of about 0.42, a focal length of about 10 mm and a working distance of about 20 mm. The detection objective lens 1022 may be of the same configuration as the illumination objective lens 1010. Objective lens with long working distances may be used as the illumination objective lens 1010 and the detection objective lens 1022, so that the illumination objective lens 1010 and the detection objective lens 1022 may be employed at a suitable distance away from the specimen. This may be especially important for non-contact ocular imaging, so that imaging device can be positioned at a finite working distance away a patient's eye. The detection objective lens 1022 may be arranged in an orthogonal fashion in the first collection arm, in other words, the first detection optics arrangement. The first collection arm may be used for fluorescence overlaid angle imaging. The detection objective lens 1022 may have the same NA as the illumination objective lens 1010, in order to achieve isotropic resolution. The microscope 1014 may be for example, a mini USB digital microscope. The microscope 1014 may be defined for the positioning of the sample, in other words, the specimen 1020. The microscope 1014 may also be configured for the visualization of the area of beam interrogation. The collected signal using the detection objective lens 1022 may be further filtered using the notch filter 1226. The notch filter 1226 may have a filter wavelength of 488 nm. The collected signal may be imaged onto the first imaging sensor 1026. The first imaging sensor may be, for example, a low light sensitive scientific Complementary Metal-Oxide-Semiconductor camera (sCMOS) camera. The first imaging sensor may have a maximum frame rate of 30 frames per second with 2560×2120 pixels of 6.5 microns size. The detection tube lens 1024 may be infinity-corrected and have the same specifications as the illumination tube lens 1008. Although the prototype has a sCMOS camera as the imaging sensor, the imaging sensor may include or may be other forms of imaging sensors. The second detection optics arrangement may be used for corneal imaging. In the second detection optics arrangement, imaging may be performed in the reflection scheme where the reflected signal from the specimen 1020 collected using the illumination objective lens 1010 may be redirected into the second imaging sensor 1116. The second imaging sensor 1116 may be for example, a monochrome digital coupled device camera (CCD). The CCD may have a resolution format of 1.3 megapixel, ⅔″ CMOS 1280×1024 resolution and 6.7 μm square pixels. The imaging lens, in other words, the focusing lens 1114, may be optically coupled to the beam splitter 1110. The beam splitter 1110 may be a pellicle beam splitter having a R:T split ratio of 45:55. Although the above embodiment has a CCD camera as the imaging sensor, the imaging sensor may include or may be other forms of imaging sensors. Similarly, while the above embodiment has a pellicle beam splitter as the beam splitter, it will be understood that other forms of beam splitters may be used. The variable neutral density filter 1224 may be placed between the beam splitter 1110 and the second imaging sensor 1116, in order to control the light intensity. Depth-sensitive measurements may also be carried out by moving the illumination objective lens 1010 at micro level, using a translation stage.

The spatial resolution of the imaging device 1200 for the purpose of angle imaging using the first detection optics arrangement, may be defined by the NA of the detection objective lens 1022. The resolution value may be about 0.7 μm in free-space. When imaging through a turbid medium, the resolution value may be around 1 micron. The axial resolution of the imaging device may be approximately equal to depth of focus (1.6 μm) or less than 2 μm, which defines the depth of image that appears to be sharply in focus at one setting of the fine-focus adjustment. The field of view may be about 0.44×0.33 mm. The axial resolution may be determinable based on a NA of the illumination objective lens 1010 and may be proportional to the numerical aperture of the illumination objective lens 1010. The axial resolution may be further determinable based on a wavelength of the Bessel beam in vacuum and may be proportional to the wavelength of the Bessel beam in vacuum.

The spatial resolution of the imaging device 1200 for the purpose of angle imaging using the first detection optics arrangement, may be defined by the NA of the detection objective lens 1022. The resolution value may be about 0.7 μm in free-space. When imaging through a turbid medium, the resolution value may be around 1 micron. The axial resolution of the imaging device may be approximately equal to depth of focus (1.6 μm), which defines the depth of image that appears to be sharply in focus at one setting of the fine-focus adjustment. The field of view may be about 0.44×0.33 mm.

The scan lens 1006, also referred herein as the visible scan lens, may be a tele-centric objective. The scan lens 1006 may be desirable for laser scanning applications because of the flat imaging plane that results from its tele-centricity. The scan lens 1006 may produce geometrically correct images with minimal image distortion, as the laser beam may be scanned across the back aperture of the scan lens 1006. In a point-by-point laser scanning system, the focal spot may not necessarily coincide with the optical axis of the visible scan lens 1006. In contrast to a conventional lens that will result in serious aberrations and poor image quality, the scan lens 1006 may be designed to produce uniform spot size and optical path length at every scan position. A uniform spot size over the entire field of view (FOV) in turn translates to a high quality and uniform image. In the 4F configuration of the scan lens 1006, the illumination tube lens 1008 and the illumination objective lens 1010, the optimal scanning position may be dependent on the distance between the illumination tube lens 1008 and the illumination objective lens 1010, which are both infinity-corrected lenses. The longer the distance, the shorter the scanning position and vice versa. The visible scan lens 1006 and infinity-corrected illumination tube lens 1008 may be arranged in a 4F configuration, and the distance between the infinity-corrected illumination tube lens 1008 and the illumination objective lens 1010 is kept at 90 mm throughout the experiment, which is within the optimal distance of 70 mm to 170 mm as recommended by the manufacturer. The visible scan lens 1006 may be positioned such that the scanning mirror 1004 is at its eye-point, while the field aperture plane is at its focal point. The scanning mirror 1004 may be configured to sweep the focused Bessel beam in the y-direction to create a virtual light sheet at each z-plane of the 3D volumetric stack in a raster fashion. The scanning mirror 1004 may have an aperture size at least substantially equal to 10 mm. The size and intensity profile of the virtual light sheet may be controlled using software which may reside in a memory module of the personal computer 1230. The light sheet for illumination may be positioned such that it is within the depth of field of the detection objective lens 1022. The efficiency of the illumination source may be maximized because light may be concentrated only at the region of interest. In addition, image sharpness may be improved while background noise may be minimized since the specimen that is not within the objective's depth of field may not contribute to the out-of-focus blur.

The 4F configuration following the axicon lens 1016 may allow the Bessel beam to alternate between the beam phase and the ring phase when passing through the lenses. The Bessel beam will expand into a ring after some distance of propagation. The 4F configuration will focus the ring back into a beam. The expansion and focusing will continue as long as successive optics are placed in the 4F configuration. The depth of focus, z_{D}, of the imaging device 1200 may be regulated by the variable aperture 1220 before the surface of the axicon lens 1016. The depth of focus, z_{D} may be approximated by

$${Z}_{D} = \frac{{R}_{ill}}{( n - 1 ) α} ,$$

where Rᵢₗₗ is the illumination radius of the plane wave incident onto the surface of the axicon lens 1016 while n is the refractive index of the axicon lens 1016 and a is the apex angle of the axicon lens 1016. The variable aperture 1220 may determine Rᵢₗₗ which in turn may determine z_{D}. For a given numerical aperture of the illumination objective lens 1010 (NAᵢₗₗ), the larger the Rᵢₗₗ, the greater the Z_{D} and the more energy exists in the side lobes. The converse may also be true. In other words, the Bessel-like characteristics and hence the length of the beam output beam may be linear with the aperture radius. The thickness of the virtual light sheet may be proportional to

$$\frac{{λ}_{0}}{2 {NA}_{ill}} ,$$

where λ₀ is the wavelength of the illumination source in vacuum. One important factor for consideration is to use a beam that is just sufficiently long enough to cover the desired region of interest. Otherwise, the increased side lobes energy may also result in an increase in excitation on either side of the central core. The lateral resolution on the other hand, may be similar to the conventional diffraction limit of wide field microscopy and is given by

$$\frac{{λ}_{fl}}{2 {NA}_{det}} ,$$

where λ_{fl} is the fluorescence emission wavelength and NA_{det} is the numerical aperture of the detection objective lens.

In other words, the depth of focus may be determinable based on a radius of a plane wave incident onto a surface of the axicon lens 1016. The depth of focus may be further determinable based on an apex angle of the axicon lens 1016, such as inversely proportional to the apex angle. The depth of focus may be further determinable based on a refractive index of the axicon lens 1016 and may be inversely proportional to the refractive index of the axicon lens 1016 minus one.

The imaging device 1200 may be able to carry out corneal imaging when it is operated in a corneal imaging mode. The imaging device 1200 may also be able to carry out angle imaging when it is operated in an angle imaging mode. The imaging device 1200 may be configured to carry out corneal imaging and angle imaging sequentially. The corneal imaging may be carried out using a near infrared source while the angle imaging may be carried out using a laser beam having a wavelength of about 488 nm. The patient or the specimen may need to be positioned slightly differently for the corneal imaging mode and the angle imaging mode. The imaging device 1200 may be configured to use the same scanning mirror 1004 for both the corneal imaging mode and the angle imaging mode. In the corneal imaging mode, the scanning mirror 1004 may be configured to scan at a lower scanning speed than in the angle imaging mode. The imaging device may be able to achieve high repeatability and reproducibility for corneal imaging and moderate repeatability and reproducibility for angle imaging. The process of corneal imaging may take less than one minute to complete whereas the process of angle imaging of one quadrant may take about one minute. These timings may exclude the time required for aligning or positioning the specimens. The images obtained by the imaging device may be analysed in a computer system using image processing schemes or algorithms. The analysis process may take less than two minutes.

The desired specifications of the individual components of an imaging device according to various embodiments, are described in the following paragraphs. It should be noted that the described specifications are not mandatory and may be varied according to the imaging applications. Accordingly, the final specifications of the imaging device depend on the specifications of the individual components. While the individual components may be replaced with similar functioning optical components, the assembly consisting of the scanning mirror 1004, the scan lens 1006, the illumination tube lens 1008 and the illumination objective lens 1010 should satisfy the 4F configuration as discussed above.

The laser generator 1010 may be configured to provide a laser beam having a wavelength of about 488 nm. The wavelength of the laser may be intended for emission of a fluorophore, for example, fluorescein. Fluorescein is a fluorescence sample that can be used inside the eye. Fluorescein has an absorption maximum at 494 nm and an emission maximum of 521 nm (in water). Based on stokes shift, a range of excitation wavelengths between 480 to 500 nm may be used for the excitation of the fluorescein.

The FC/PC fiber collimation package including the fiber collimator 1014 and the single mode fiber 1012 may be designed for wavelengths over the whole visible spectrum of 400 to 700 nm. The fiber collimator 1014 may include a collimating lens, for example, having a numerical aperture (NA) of 0.26 and a focal length of 34.74 mm.

The axicon lens 1016 may be is anti-reflection (AR) coated for wavelengths of 425 to 675 nm. The apex angle of the axicon lens 1016 may be about 176°, in other words, axicon angle of 2°. Axicon lenses with lower axicon angle are generally preferred for creating optical setups with long working distances. Hence the apex angle of the axicon lens 1016 may fall within the range of 168° to 178°.

The scanning mirror 1004 may be AR coated for a wide range of wavelengths including the visible and the near infrared region. The scanning speed, as well as the area of the scanning, may be defined using interfacing software. The speed of scanning may increase up to 3000 mm/sec. The interfacing software may reside on a storage module in the personal computer 1230.

The illumination tube lens 1008 may be infinity-corrected. Its focal length of the illumination tube lens 1008 may be about 200 mm and its working distance may be about 148 mm.

The scan lens 1006 may be infinity-corrected. Its focal length may be at least substantially equal to 70 mm and its working distance may be at least substantially equal to 54 mm.

The illumination objective lens 1010 may have a long working distance. The illumination objective lens 1010 may be a plan apochromat lens having a magnification of about 20 times, NA of about 0.42, focal length of about 10 mm and a working distance of about 20 mm. The detection objective lens 1022 may ideally have the same specifications as the illumination objective lens 1010. These objective lenses may be replaced with other long working distance objective lenses. The specifications of the illumination objective lens 1010 may be at least substantially in the range of: magnification=10× to 40×; NA=0.2 to 0.5; focal length=8 to 25 mm; and working distance=15 to 30 mm. The illumination objective lens 1010 may be a plan apochromat lens.

The first imaging sensor 1026 may be a low light sensitive sCMOS camera with a reasonably good frame rate. But the detector specifications are not limited to it. The specifications of the first imaging sensor 1026 may be at least substantially in the range of: 8 or 16-bit resolution; 5 to 8 megapixels; frame rate of about 20 to 50 frames per second, high quantum efficiency of preferably above 60%.

The second imaging sensor 1116 may be a relatively low light sensitive camera having a frame rate of above 15 frames per second.

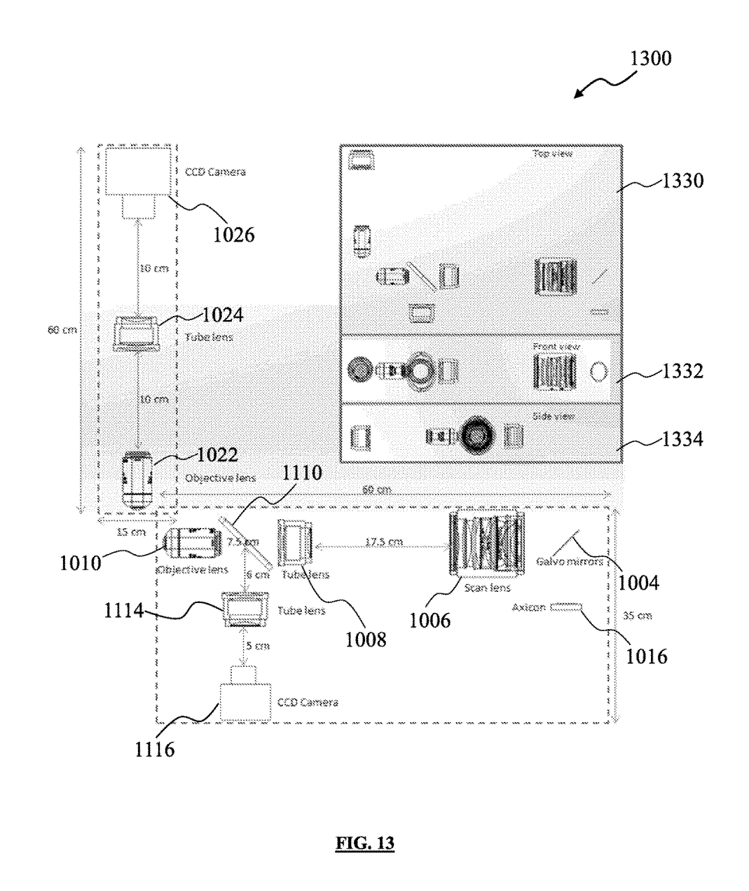

FIG. 13 shows a schematic diagram 1300 of a prototype of an imaging device according to various embodiments. The prototype imaging device may be similar to any one of the imaging devices 100A, 100B, 1000, 1100 and 1200. The prototype imaging device is used to obtain the experimental images described in the following paragraphs. The prototype imaging device has a spatial resolution of at least substantially equal to 1 micron; an axial resolution of at least substantially equal to the depth of focus; and a field of view of at least substantially equal to 0.44×0.33 mm. The axial resolution is approximately 1.6 μm The depth of field of the imaging device may depend on the maximum numerical aperture of the ring formed by the axicon and the thickness of the ring. The depth of field is approximately 50-60 μm. The imaging device is able to image a cornea at a spatial resolution of at least substantially in a range of 0.7 μm to 1 μm The prototype imaging device may have individual components with specifications as described above. The scanning mirror 1004 is a 2-dimensional (2-axis) galvanometer mirror purchased from Cambridge Technology (Watertown, Mass.). The scanning mirror 1004 has an aperture size of 10 mm and a scanning speed of up to 3000 mm per second. The scan lens 1006 was purchased from ThorLabs Inc. (Newton, N.J.). The illumination objective lens 1010 was purchased from Mitutoyo Corp., (Tokyo, Japan). The first imaging sensor 1026 is a low light sensitive scientific Complementary Metal-Oxide-Semiconductor camera (sCMOS) camera purchased from Andor (Belfast, Northern Ireland, UK). The first imaging sensor 1026 has a resolution of 16-bit and 5 megapixels. The second imaging sensor 1116 is a monochrome digital CCD purchased from PixeLINK (Ottawa, Canada). The second imaging sensor 1116 has a resolution format of 1.3 megapixel, ⅔″ CMOS 1280×1024 resolution and 6.7 μm square pixels. The prototype imaging device may form a light sheet by scanning a focused Bessel-beam. The Bessel beam may be formed by illuminating a 176° apex angle axicon lens 1016 with a Gaussian beam output of a laser. The Bessel beam may be collimated and directed into the illumination objective lens 1010 via a scanning mirror 1004. The scanning mirror 1004 may be a galvanometer driven x-y scanner. Images may be generated by raster scanning the scanning mirror 1004. The scan lens 1006 may be positioned such that the scanning mirror 1004 is at its eye-point while the field aperture plane is at its focal point. Since the illumination objective lens 1010 is infinity-corrected, an illumination tube lens 1008 may be positioned to re-collimate the excitation light. The infinity-corrected illumination tube lens 1008 may be paired with a visible scan lens 1006 (CLS-SL). The illumination tube lens 1008 may relay the scan plane of a laser scanning imaging system to the back aperture of the illumination objective lens 1010. The detection objective lens 1022 may be placed with its axis orthogonal to the sample plane. The focusing lens 1024 which may be a regular tube lens, may be used to form an image of the fluorescent structures onto the first imaging sensor 1026. The first imaging sensor 1026 may be a CMOS sensor or may be sCMOS camera. The spatial resolution of the imaging device may be defined by the numerical aperture of the detection objective lens 1022. The resolution value is about 700 nm in free-space. The resolution value may be around 1 micron for imaging in turbid medium, such as an eye. The axial resolution of the imaging device may be approximately equal to depth of focus of about 1.6 μm, which defines the depth of image that appears to be sharply in focus at one setting of the fine-focus adjustment. The field of view may be about 0.44×0.33 mm. In the corneal imaging scheme, the axicon lens 1016 may be optional. The collimated Gaussian beam may be incident on the scanning mirror 1004 from the right-hand side and may be scanned across the corneal surface using the scan lens 1006, the illumination tube lens 1008 and the illumination objective lens 1010. The reflected beam may be collected using the same objective and then directed into the second imaging sensor 1116 using a pellicle beam-splitter. The second imaging sensor may be a CCD camera. The stitching of images along the two-dimensional corneal surface and subsequently at different depth may be performed using an image processor. The image processor may be configured to run an image processing algorithm. The image processor may be part of a personal computer. The image processor may be configured to receive two-dimensional images from any one of the first imaging sensor 1026 or the second imaging sensor 1116. The image processor may be configured to convert the plurality of two-dimensional images obtained at different depths of the specimen 1020 into a three-dimensional reconstruction of the specimen 1020. The first imaging sensor and the second imaging sensor are high-end detector cameras. The prototype imaging device includes a motorized stage that has a big controller unit. For translating the prototype imaging device to a clinical instrument, the motorized stage and the imaging sensors may be miniaturized as the imaging device may not require such high precision movement and high sensitivity detection. The imaging device has the advantages of being able to perform imaging without contacting the specimen, and as such, may be easily translated to a clinical standard. It is also able to provide imaging at a higher resolution than other clinical imaging instruments.

In the following, imaging experiments using an imaging device according to various embodiments will be described.

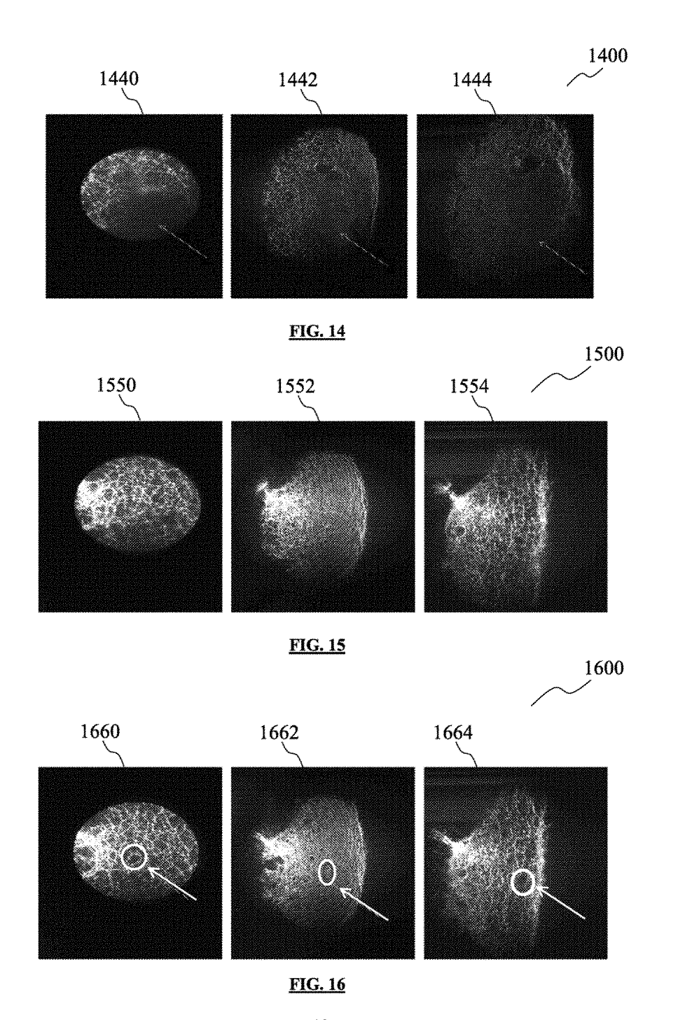

FIG. 14 shows a series of images 1400 obtained by illuminating a specimen with a Gaussian laser beam using an imaging device according to various embodiments. The specimen was partially obscured by an opaque obstacle. The obscurations in the images 1440, 1442 and 1444, caused by the opaque obstacle, are indicated by respective arrows in the images. The image 1440 corresponds to frame 1 where the image is taken from before the focal plane. The image 1442 corresponds to frame 180 where the image is taken near the focal plane. The image 1444 corresponds to frame 350 where the image is taken after the focal plane.

FIG. 15 shows a series of images 1500 obtained by illuminating a specimen with a Bessel beam using an imaging device according to various embodiments, wherein the specimen has no obstacles. The image 1550 corresponds to frame 1 where the image is taken from before the focal plane. The image 1552 corresponds to frame 180 where the image is taken near the focal plane. The image 1554 corresponds to frame 350 where the image is taken after the focal plane. As can be seen from FIG. 15, the images 1550, 1552 and 1554 obtained with a Bessel beam illumination, are at least substantially clearer than the images 1440, 1442 and 1444 obtained using a Gaussian beam illumination.

FIG. 16 shows a series of images 1600 obtained by illuminating a specimen with a Bessel beam using an imaging device according to various embodiments, wherein the specimen is partially obscured by an opaque obstacle. The image 1660 corresponds to frame 1 where the image is taken from before the focal plane. The image 1662 corresponds to frame 180 where the image is taken near the focal plane. The image 1664 corresponds to frame 350 where the image is taken after the focal plane. The position of the opaque obstacle is marked out on the images by a circle and an arrow. As can be seen from FIG. 16, the region of the specimen behind the opaque obstacle is clearly visible in the images 1660, 1662 and 1664. The Bessel beam is able to circumvent and reconstruct behind the obstacle to provide clear, unobstructed images. This feature may be very useful in imaging specimens when there are opaque obstacles, such as corneal pathology. When the cornea degrades, parts of the cornea may become opaque, for example, due to calcium deposits at the limbus. As shown in the images obtained in FIG. 16, the imaging device may overcome the difficulties in imaging a degraded cornea.

In an experiment, one randomly selected eye of a New Zealand white rabbit was applied with 2% fluorescein sodium eye drops four hours prior to the start of the experiment. The fluorescein sodium eye drops were applied at an interval of 5 minutes for 15 minutes, and the excess was washed off with saline solution. The untreated eye of the rabbit served as a control. The imaging device is then used to image both the treated and untreated eyes of the rabbit.

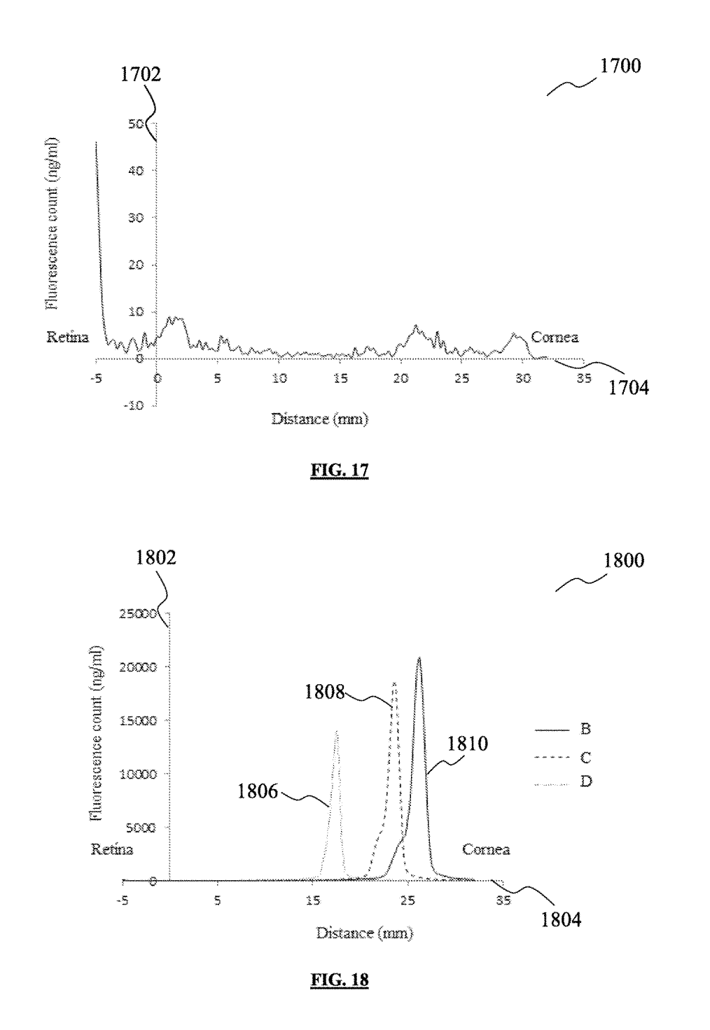

FIG. 17 shows a graph 1700 showing the background level of fluorescence count in the untreated eye of the New Zealand white rabbit. The graph 1700 includes a vertical axis 1702 indicating the fluorescence count in nanograms per millilitres (ng/ml); and a horizontal axis 1704 indicating distance in millimetres (mm). The horizontal axis 1704 shows a position in the eye, wherein the distance of about −5 to 0 mm refers to the retina while the distance of about 30 mm refers to the cornea.

FIG. 18 shows a graph 1800 showing the fluorescence count in the eye of the rabbit in which fluorescein is applied. The graph 1800 includes a vertical axis 1802 indicating the fluorescence count in ng/ml; and a horizontal axis 1804 indicating distance in mm. The horizontal axis 1804 shows a position in the eye, wherein the distance of about −5 to 0 mm refers to the retina while the distance of about 30 mm refers to the cornea. The spatial resolution of the imaging device was about 0.7 μm. The graph 1800 further includes a first plot 1810, labeled as “B”; a second plot 1808, labeled as “C”; and a third plot 1806, labeled as “D”. The first plot 1810, the second plot 1808 and the third plot 1806 represent three different independent scans targeted at different depths of the eye. The graph 1800 shows that fluorescein is able to reach the anterior chamber of the eye via eye drops application. In the high resolution corneal imaging mode, the imaging device is able to image the collagen fibers with sufficient resolution for disease detection and monitoring.

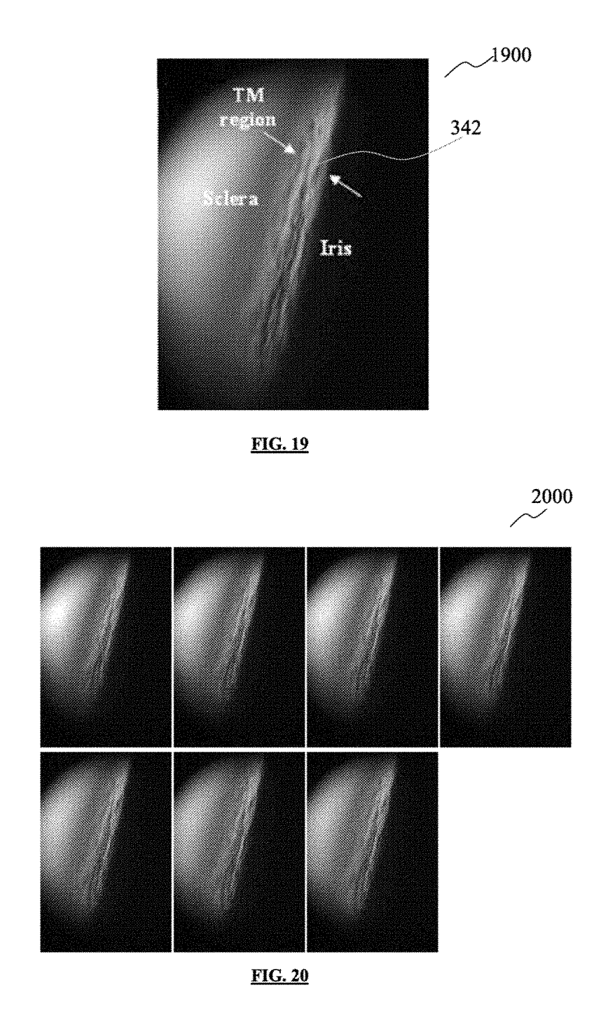

FIG. 19 shows an image 1900 showing a representative image obtained using an imaging device according to various embodiments, the representative image showing an angle region of a porcine eye sample. A proof-of-concept experiment was carried out on a fluorescein-injected porcine eye sample that was inserted into a custom eye holder to image the trabecular meshwork (TM) region. The image 1900 shows that the TM 342 region of the porcine eye can be clearly visible. Porcine eyes were selected to test the imaging device as they are easily available and the surfaces of their TM structures are similar to that of the human eyes.

FIG. 20 shows a series of images 2000 of the angle region, obtained at different depths. The wavelength of the laser beam is about 488 nm while the beam spot size is about 1.1-1.8 μm The scanned area is about 3 mm×0.35 mm.

FIG. 21 shows eye images 2100 obtained using Bessel beam based digital light sheet microscopy, according to various embodiments. The eye images 2100 includes a first image 2102 obtained using a monochrome imaging sensor; a second image 2104 obtained using a color CCD as the imaging sensor; and a third image 2106 obtained using a static light sheet.

The imaging device may be able to constantly monitor the aqueous humour flow rate with the use of exogenous agent such as fluorescein, and may be able to monitor the drug delivery route in the anterior and posterior chamber of the eye, for treatment of ocular diseases. The dosage of the drug administered can therefore be optimized for individual patients. As such, the imaging device may be used as an instrument for the management of glaucoma and its clinical subtypes.

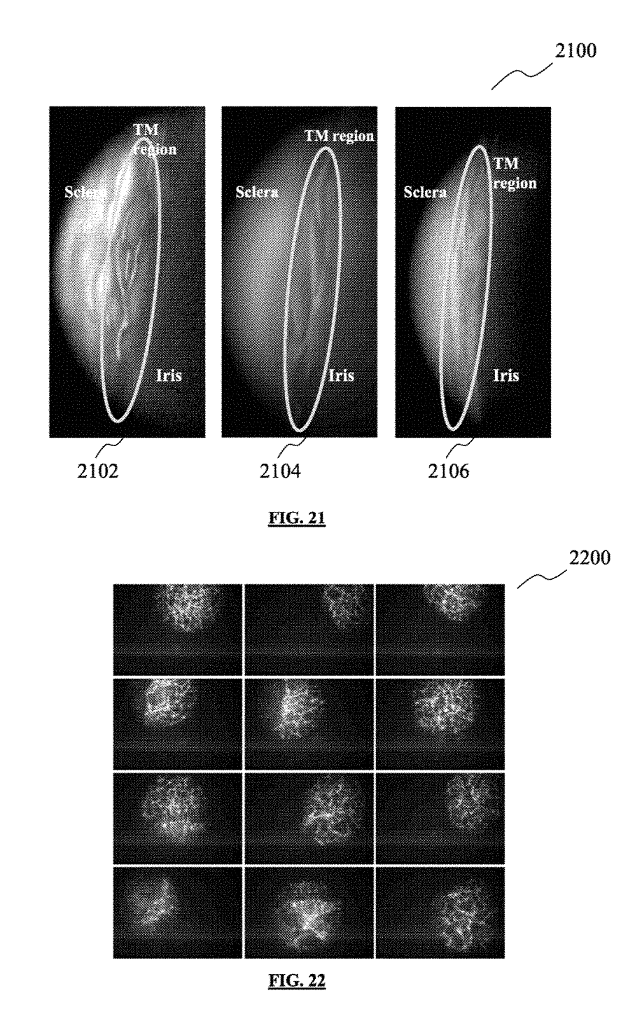

FIG. 22 shows a series of snapshots 2200 of a porcine cornea. The porcine cornea was scanned over a 25 mm×25 mm area without automation.

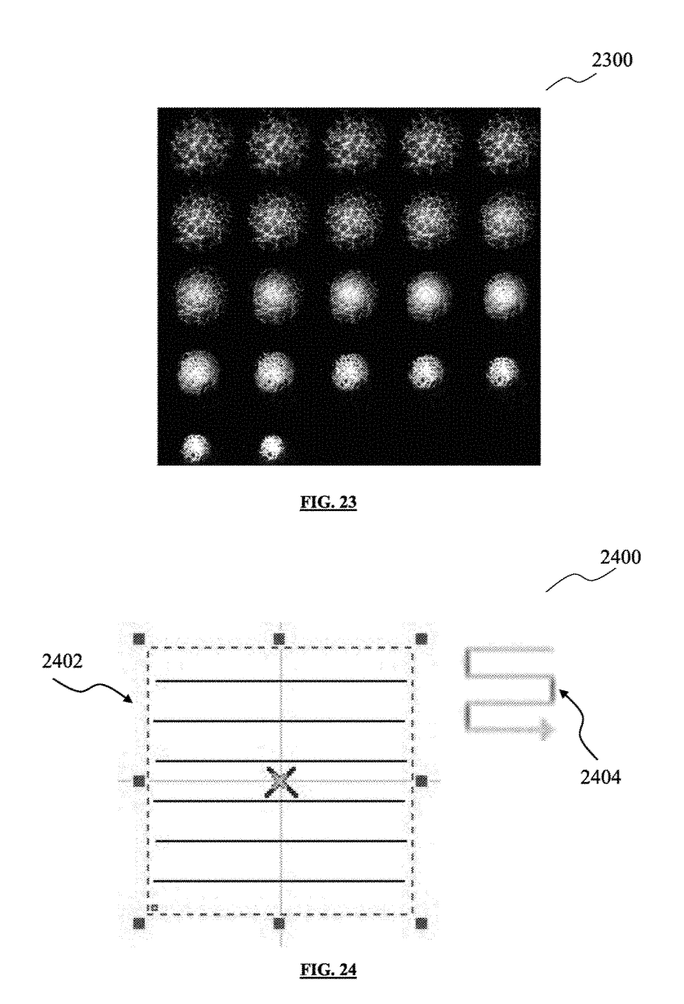

FIG. 23 shows a series of porcine corneal images 2300 at different depths. The spatial resolution of the imaging device is about 0.7 μm. This high resolution corneal imaging mode can image the collagen fibers with sufficient resolution for disease detection and monitoring. For example, the imaging device may be used to study or monitor the regeneration of the corneal sub basal nerves after laser-assisted in situ keratomileusis (LASIK). The imaging device may be used to detect abnormalities in the extracellular matrix of the cornea, hence detecting or identifying inflammatory and non-inflammatory diseases of the cornea. Unlike in vivo confocal microscopy, it is non-contact.

FIG. 24 shows a diagram 2400 showing the 25×25 mm grid 2402 drawn on the laser marking software and the direction of scan 2404.

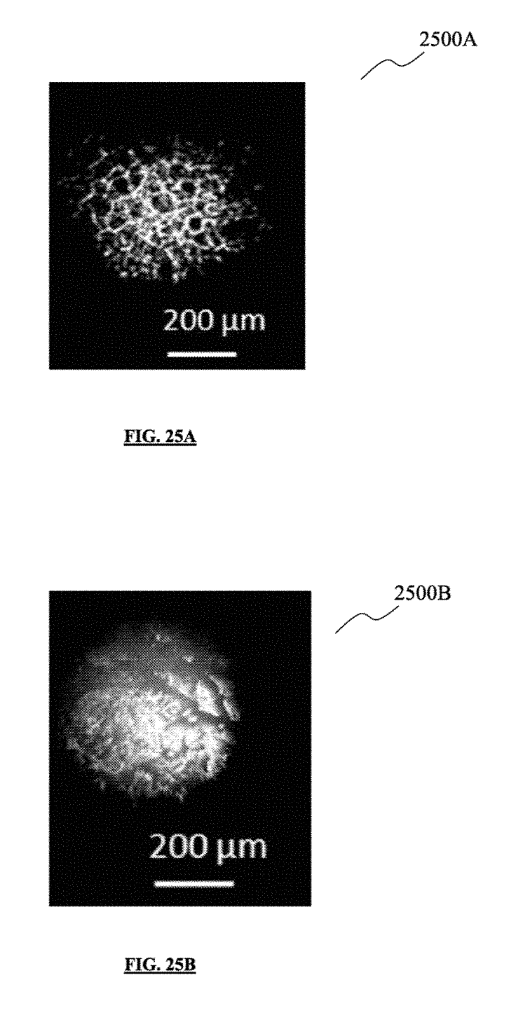

FIG. 25A shows an image 2500A of a New Zealand white rabbit's healthy cornea. The image was obtained using an imaging device according to various embodiments. To validate the clinical significance of the imaging device, the imaging device was used to image the white rabbit's cornea before infection and after infection with Pseudomonas.

FIG. 25B shows an image 2500B of the white rabbit's cornea of FIG. 25A, 10 days after the infection. The imaging of the infected eye was performed 10 days after the infection. Pseudomonas multiplied rapidly and crowded out the host tissues, hence disrupting the normal physiology of the eye. The more densely populated epithelium and higher reflectivity of the figure can be associated with hallmarks of bacteria keratitis such as loss of cornmeal transparency, peripheral epithelial edema, and deep stroma abscesses.

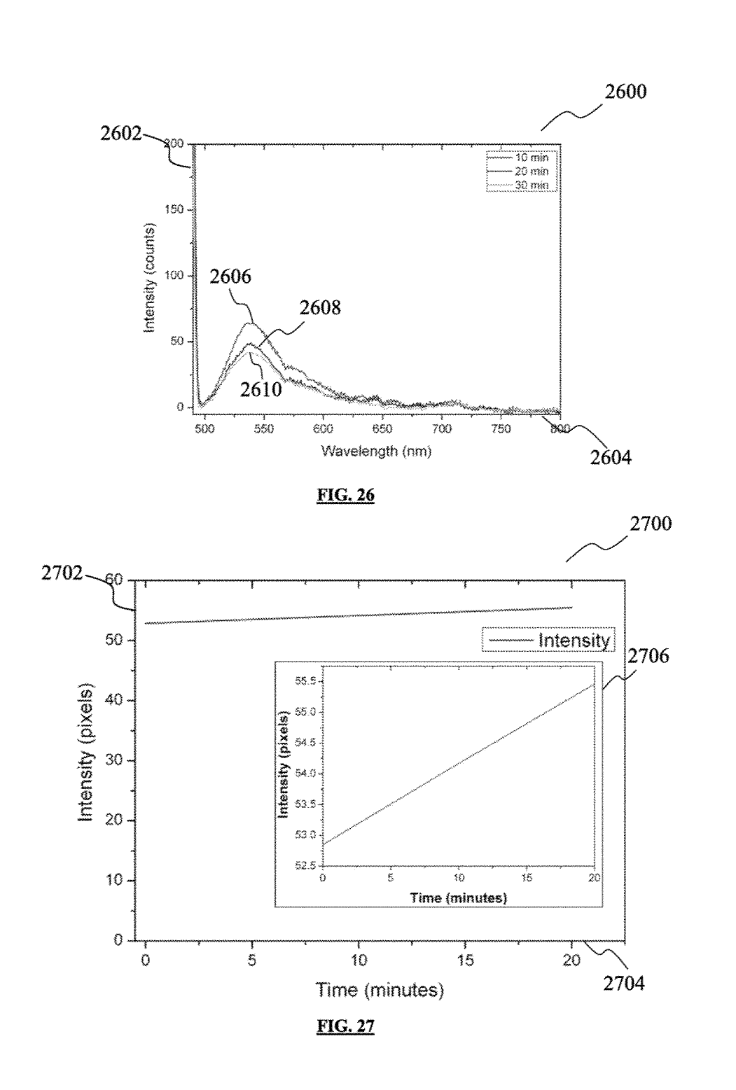

FIG. 26 shows a graph 2600 showing how a fluorescent intensity on the corneal surface of an eye varies with a Bessel beam wavelength. The fluorescent intensity was recorded by a spectrometer. Fluorescein was applied to the eye that is being examined. The graph 2600 includes a vertical axis 2602 indicating the fluorescent intensity in counts, and a horizontal axis 2604 indicating wavelength of the Bessel beam in nanometers (nm). The graph 2600 further includes a first plot 2606 showing the fluorescent level 10 minutes after the fluorescein is applied; a second plot 2608 showing the fluorescent level 20 minutes after the fluorescein is applied; and a third plot 2610 showing the fluorescent level 30 minutes after the fluorescein is applied. As can be seen from the graph 2600, the fluorescent intensity is the highest when the Bessel beam wavelength is about 520 nm.

FIG. 27 shows a graph 2700 showing the fluorescent level in the anterior chamber of the eye of FIG. 26. The graph 2700 includes a vertical axis 2702 indicating intensity in pixels; and a horizontal axis 2704 indicating time in minutes. FIG. 27 further includes an inset graph 2706 that shows the graph 2700 with the vertical axis 2702 truncated to vary from 52.5 to 56. Fluorescein may be applied to the aqueous humour so that the flow of the aqueous humour may be monitored by observing the fluorescent intensity in different parts of the eye. In other words, the imaging device may be used to analyse the aqueous humour flow rate using fluorescence quantification method. For example, in an eye suffering from glaucoma, the flow of the aqueous humour from the posterior chamber to the anterior chamber may be blocked. By observing the fluorescent intensity in the anterior chamber after a predetermined time duration, a clinician may be able to determine if the aqueous humour flow to the anterior chamber is normal. As such, the imaging device may be used to monitoring the flow rate of aqueous humor in the eye, and may be valuable as an instrument for managing glaucoma and its clinical subtypes.

In the above-described experiments, imaging devices and methods for imaging specimens are demonstrated. The method may include performing Bessel beam light sheet fluorescence microscopy (BB-LSFM) by combining Bessel beam-excited fluorescence with the orthogonal illumination of light sheet microcopy. With the ex-vivo imaging of the whole porcine eye and subsequent in-vivo trials on New Zealand white rabbits and non-human primates, the high performance of BB-LSFM as compared to the current state-of-the-art imaging techniques in maintaining good signal and high spatial resolution deep inside the trabecular meshwork structures, in acquisition speed, and in low phototoxicity are demonstrated. Together, these properties make the method for imaging specimens, a non-contact and non-invasive approach to objectively evaluate the iridocorneal angle region of glaucoma patients.

According to various embodiments, the imaging device may provide a non-contact and non-invasive optical probe system for the high resolution imaging and characterization of the various layers of the corneal. Depth-sensitive measurements may be carried out by moving the detection objective lens at the micro level using a translational stage. The low photo-bleaching and low photodamage characteristics of using the imaging device make it ideal for ocular imaging.

According to various embodiments, the imaging device may provide a non-contact and non-invasive optical probe system for the high resolution imaging and characterization of the trabecular meshwork and anatomical structures of the aqueous outflow system. Currently, there is no commercially available clinical instrumentation for imaging structures of the trabecular meshwork region of the eye with sufficient resolution for either diagnosing or following up the progression of angle-closure glaucoma. The imaging device may image the aqueous outflow system inside the eye using conventional static light sheet and Bessel-beam based digitally scanned light sheet microscopic techniques. The image contrast and anatomical discrimination in the optical slices obtained may be enhanced by overlaying the desired region with fluorescence dye distribution profile. The imaging device is therefore a useful instrument for the management of glaucoma and its clinical subtypes.

According to various embodiments, the method employs a non-contact in vivo imaging principle for imaging the iridocorneal region of an eye. Unlike gonioscopy, there is no contact between the eye and a goniolens or a prism or coupling gel. Gonioscopy may be a painful process, as the goniolens has to be pressed against the eyeball to minimize total internal reflection. Also, the method as the advantage that the patient may be in a sitting position, for both angle imaging and corneal imaging. The imaging device used in the method may be simple to operate, such that only basic training may be required to handle the imaging device. The imaging device may even be fully automated and as such, the method may not require expert operators to operate the imaging device.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced. It will be appreciated that common numerals, used in the relevant drawings, refer to components that serve a similar or the same purpose.

1. An imaging device comprising:
a Bessel beam generator configured to provide a Bessel beam;
a scanning mirror configured to scan the Bessel beam across a two-dimensional plane;
a scan lens configured to receive the Bessel beam from the scanning mirror, a centre of the scan lens being at least substantially a focal length of the scan lens away from the scanning mirror;
an illumination tube lens configured to receive the Bessel beam from the scan lens, a centre of the illumination tube lens being at least substantially a sum of the focal length of the scan lens and a focal length of the illumination tube lens away from the centre of the scan lens;
an illumination objective lens positioned in direct line-of-sight to a specimen, the illumination objective lens configured to receive the Bessel beam from the illumination tube lens and further configured to illuminate the specimen with the Bessel beam, wherein a centre of the illumination objective lens is at least substantially the focal length of the illumination tube lens away from the centre of the illumination tube lens; and
a detection optics arrangement configured to receive a reflected beam from the specimen.

2. The imaging device of claim 1, wherein the reflected beam is a reflection of the Bessel beam, by the specimen.

3. The imaging device of claim 1, wherein the Bessel beam generator comprises
a laser generator configured to generate a Gaussian beam;
a collimator coupled to the laser generator for receiving the Gaussian beam and configured to collimate the Gaussian beam;
an aperture for receiving the collimated Gaussian beam from the collimator and passing through a further collimated Gaussian beam, the aperture being variable for adjusting a depth of focus of the imaging device; and
an axicon lens for converting the further collimated Gaussian beam into the Bessel beam.

4. The imaging device of claim 3, wherein the Bessel beam generator further comprises a single mode fiber for coupling the laser generator to the collimator.

5.-6. (canceled)

7. The imaging device of claim 3, wherein the Bessel beam generator further comprises a further collimator configured to collimate the Bessel beam.

8.-10. (canceled)

11. The imaging device of claim 3, wherein the detection optics arrangement comprises
a detection objective lens positioned at least substantially orthogonal to the illumination objective lens, the detection objective lens configured to receive the reflected beam from the specimen;
a detection tube lens coupled to a back aperture of the detection objective lens for receiving the reflected beam from the detection objective lens; and
an imaging sensor configured to receive the reflected beam from the detection tube lens.

12. The imaging device of claim 11, wherein the detection optics arrangement further comprises a notch filter positioned between the detection objective lens and the detection tube lens.

13-15. (canceled)

16. The imaging device of claim 11, wherein the imaging device has a lateral resolution determinable based on a numerical aperture of the detection objective lens.

17. (canceled)

18. The imaging device of claim 11, wherein the imaging device has a lateral resolution determinable based on an emission wavelength of a fluorophore.

19. The imaging device of claim 18, wherein the lateral resolution is proportional to the emission wavelength of the fluorophore.

20. The imaging device of claim 1, wherein the detection optics arrangement comprises
a beam splitter positioned between the illumination tube lens and the illumination objective lens, the beam splitter configured to receive the reflected beam from the specimen through the illumination objective lens and further configured to partially reflect the reflected beam in a direction at least substantially orthogonal to the reflected beam;
a focusing lens configured to receive the partial reflection of the reflected beam from the beam splitter; and
an imaging sensor configured to receive the partial reflection of the reflected beam from the focusing lens.

21. The imaging device of claim 20, wherein the detection optics arrangement further comprises a variable neutral density filter positioned between the focusing lens and the imaging sensor.

22-29. (canceled)

30. The imaging device of claim 1, wherein the Bessel beam has a wavelength at least substantially corresponding to an excitation wavelength of a fluorophore.

31-33. (canceled)

34. The imaging device of claim 1, wherein the scanning mirror is configured to scan in a raster scanning pattern.

35-51. (canceled)

52. The imaging device claim 1, wherein the depth of focus is determinable based on a radius of a plane wave incident onto a surface of the axicon lens.

53. The imaging device of claim 3, wherein the depth of focus is determinable based on an apex angle of the axicon lens.

54. (canceled)

55. The imaging device of claim 1, wherein the depth of focus is determinable based on a refractive index of the axicon lens.

56. (canceled)

57. The imaging device of claim 1, wherein the imaging device has an axial resolution determinable based on a numerical aperture of the illumination objective lens.

58. (canceled)

59. The imaging device of claim 1, wherein the imaging device has an axial resolution determinable based on a wavelength of the Bessel beam in vacuum.

60-64. (canceled)

65. A method for imaging a specimen, the method comprising:
generating a Bessel beam using a Bessel beam generator;
scanning the Bessel beam across a two-dimensional plane using a scanning mirror;
receiving the Bessel beam from the scanning mirror using a scan lens, a centre of the scan lens being at least substantially a focal length of the scan lens away from the scanning mirror;
receiving the Bessel beam from the scan lens using an illumination tube lens, a centre of the illumination tube lens being at least substantially a sum of the focal length of the scan lens and a focal length of the illumination tube lens away from the centre of the scan lens;
receiving the Bessel beam from the illumination tube lens and illuminating the specimen with the Bessel beam using an illumination objective lens, the illumination objective lens being positioned in direct line-of-sight to the specimen, wherein a centre of the illumination objective lens is at least substantially the focal length of the illumination tube lens away from the centre of the illumination tube lens; and
receiving a reflected beam from the specimen using a detection optics arrangement.
